(19) United States
(12) Patent Application Publication
(10) Pub. No.: US 2026/0041339 A1
(43) Pub. Date: Feb. 12, 2026

(54) NON-INVASIVE MEASUREMENT OF ENDOGENOUS S-NITROSOTHIOLS

(71) Applicants: University Hospitals Cleveland Medical Center, Cleveland, OH (US); NNOXX, INC., Seattle, WA (US)

(72) Inventors: Jonathan s. Stamler, Cleveland, OH (US); Evan Peikon, Manson, WA (US)

(21) Appl. No.: 19/364,250

(22) Filed: Oct. 21, 2025

Related U.S. Application Data
(63) Continuation of application No. 17/885,843, filed on Aug. 11, 2022.
(60) Provisional application No. 63/232,686, filed on Aug. 13, 2021.
(60) Provisional application No. 63/289,470, filed on Dec. 14, 2021.
(60) Provisional application No. 63/339,871, filed on May 9, 2022.
(60) Provisional application No. 63/347,661, filed on Jun. 1, 2022.

(51) Int. Cl. A61B 5/145; A61B 5/00; A61B 5/0205; A61B 5/026; A61B 5/1455

(52) U.S. Cl. CPC A61B 5/14546; A61B 5/0205; A61B 5/0261; A61B 5/14556; A61B 5/4833; A61B 5/4866; A61B 5/4884; A61B 5/7246; A61B 5/7257; A61B 5/7475

(57) ABSTRACT

Systems and methods are provided for non-invasive measurement of endogenous S-nitrosothiols and related measurements thereof. One or more sensors non-invasively measures a set of one or more biometric parameters within a region of interest of a subject to provide a time series of measurements for each of the set of biometric parameters. A medium stores machine-readable instructions that are executable by an associated processor to perform processing comprising receiving the time series of measurements of the biometric parameter, generating, using a predictive model, a value representing an endogenous S-nitrosothiol content of tissue within the region of interest from the time series of measurements of the biometric parameter, and providing, by a user interface, the value representing the endogenous S-nitrosothiol content of tissue within the region of interest to a user.

### RELATED APPLICATION DATA

This is a continuation of U.S. application Ser. No. 17/885,843 filed on Aug. 11, 2022, which claims priority under 35 U.S.C. § 119(e) to U.S. Ser. No. 63/232,686, filed on Aug. 13, 2021; U.S. Ser. No. 63/289,470, filed on Dec. 14, 2021; U.S. Ser. No. 63/339,871, filed on May 9, 2022; and U.S. Ser. No. 63/347,661 filed on Jun. 1, 2022, the contents of which are all incorporated by reference herein.

### BACKGROUND OF THE DISCLOSURE

### Field of the Disclosure

This invention relates to diagnostic systems, and more particularly, to non-invasive measurement of endogenous S-nitrosothiols.

### Background Information

Nitric oxide (NO) has been associated with many physiological effects, among them, smooth muscle relaxation, vasodilation, inflammation responses, and inhibition of platelet adhesion and aggregation. Finding the natural reservoirs of NO and finding ways to regulate the levels of biologically available NO and its alternative bioactive forms could provide a means to control these physiological effects. Nitric oxide (NO) and S-nitrosothiols (SNOs) are carried by hemoglobin together with oxygen. SNOs are a bioactive form of NO and the only endogenously active form of NO that can survive in blood, as NO itself cannot escape from red blood cells. SNO is released from hemoglobin in tissues, for example, when under hypoxia or during exercise, to dilate blood vessels and thereby oxygenate tissues. SNO released from RBCs thus controls microvascular blood flow in tissues and without this SNO tissues cannot oxygenate (Zhang PNAS 2015; Premont Circ Res 2019). SNO levels are thus a key component of VO2 (volume of oxygen consumed by tissues). While non-invasive means are available to detect oxygenated hemoglobin, no such means are available for detection of endogenous levels of NO or SNO.

### SUMMARY OF THE DISCLOSURE

In one example, a system includes a set of at least one sensor configured to non-invasively measure a biometric parameter within a region of interest of a subject and provide a time series of measurements of the biometric parameter and at least one processor in communication with the sensor. At least one non-transitory computer readable medium stores machine-readable instructions that, when executed by the at least one processor, cause the at least one processor to perform processing that includes receiving the time series of measurements of the biometric parameter, generating, using a predictive model, a value representing an endogenous S-nitrosothiol content of tissue within the region of interest from the time series of measurements of the biometric parameter, and providing, by a user interface, the value representing the endogenous S-nitrosothiol content of tissue within the region of interest to a user.

In another example, a method is provided for non-invasive measurement of endogenous S-nitrosothiols. A biometric parameter is measured non-invasively within a region of interest of a subject by at least one sensor to provide a time series of measurements of the biometric parameter. A value representing an endogenous S-nitrosothiol content of tissue within the region of interest is generated from the time series of measurements of the biometric parameter. The value representing the endogenous S-nitrosothiol content of tissue within the region of interest is stored in a non-transitory computer readable medium. In one aspect, the value of endogenous S-nitrosothiol is predictive of the rate of reoxygenation of the tissue.

In a further example, another method is provided for non-invasive measurement of endogenous S-nitrosothiols. Blood volume and oxygen saturation within a region of interest of a subject are measured non-invasively by at least one sensor to provide a time series of oxygen saturation measurements and a time series of blood volume measurements. A linear relationship between the time series of blood volume measurements and the time series of oxygen saturation measurements is determined via a predictive model. A value representing an endogenous S-nitrosothiol content of tissue within the region of interest is determined from the time series of oxygen saturation measurements and the time series of blood volume measurements from the determined linear relationship. The value representing the endogenous S-nitrosothiol content of tissue within the region of interest is stored in a non-transitory computer readable medium.

In one embodiment, the disclosure provides a method of determining the presence of or risk of dementia or loss of cognitive function in a subject. The method includes measuring the subject's nitric oxide levels during exercise to calculate a UO2 measurement, wherein a low UO2 measurement relative to a subject not having dementia is indicative of a risk of dementia or loss of cognitive function. In one aspect, the dementia or loss of cognitive function is associated with Alzheimer's Disease.

In a further embodiment, the disclosure provides a method of determining an improvement in dementia or cognitive function in a subject. The method includes measuring the subject's nitric oxide levels during exercise using a UO2 measurement at a first time point and a second time point, wherein the second time point is a time following an exercise regimen after measurement at the first time point, wherein a low UO2 measurement relative to a subject not having dementia is indicative of a dementia or loss of cognitive function at the first time point and an increase in the UO2 measurement at the second time point relative to the first time point is indicative of an improvement in the dementia or cognitive function in the subject. In one aspect, the dementia or loss of cognitive function is associated with Alzheimer's Disease.

### BRIEF DESCRIPTION OF THE DRAWINGS

FIG. 1 illustrates an example of a system for generating a value representing an endogenous S-nitrosothiol content of tissue within a region of interest of a subject;

FIG. 2 illustrates another example of a system for generating a value representing an endogenous S-nitrosothiol content of tissue within a region of interest of a subject;

FIG. 3 is a chart illustrating a PNO level for a patient during exercise;

FIG. 4 is a chart illustrating a VO2 level for a patient during exercise;

FIG. 5 depicts a chart of a time series of UO2 measurements and a time series of VO2 measurements for an athlete operating a full-body exercise bike using one sensor for recording blood volume and oxygen saturation;

FIG. 6 depicts a chart of a time series of UO2 measurements and a time series of VO2 measurements for an athlete operating a full-body exercise bike using one sensor for recording blood volume and oxygen saturation;

FIG. 7 depicts a chart of an athlete's MAX-NO Power, recorded weekly over a six-month period;

FIG. 8 depicts a chart illustrating a relationship between an athlete's MAX-NO Endurance and their critical power, recorded over a six-week period, as a scatterplot;

FIG. 9 illustrates one example of a method for generating a value representing an endogenous S-nitrosothiol content of tissue within a region of interest of a subject;

FIG. 10 illustrates another example of a method for generating a value representing an endogenous S-nitrosothiol content of tissue within a region of interest of a subject;

FIG. 11 illustrates a further method for generating a value representing an endogenous S-nitrosothiol content of tissue within a region of interest of a subject;

FIG. 12 depicts metrics and use cases for a professional sports platform using (NO);

FIG. 13 depicts a bar graph of athlete recovery measure as Max (NO)-Recovery Level at three set numbers;

FIG. 14 illustrates personal nitric oxide (PNO) over time and (NO)-regeneration over time;

FIG. 15 depicts a graph demonstrating the effects of exercise on muscle oxygenation (SmO2) and s-nitrosothiols (PNO);

FIG. 16 depicts a graph demonstrating the effects of exercise on muscle oxygenation (SmO2) and s-nitrosothiols (PNO) with a (+) correlation nested within a (−) correlation as well as a (+) correlation nested within a (+) correlation;

FIG. 17A depicts representative tracings from a control βOC93 mouse and a corresponding βOC93A mutant animal;

FIG. 17B depicts basal pO2 in gastrocnemius muscle from a control βOC93 mouse and a corresponding βOC93A mutant animal;

FIG. 17C depicts a rate of post-occlusion recovery in muscle pO2 from a control βOC93 mouse and a corresponding βOC93A mutant animal;

FIG. 18A depicts SNO-Hb isolated from fresh arterial blood for a patient group;

FIG. 18B depicts FeNO levels for a patient group;

FIG. 18C depicts total HbNO for a patient group;

FIG. 18D depicts a ratio of SNO to total HbNO for a patient group;

FIG. 19A depicts representative near-infrared sensor measurements of the recovery of Hb oxygenation over time in a healthy control and a PAD patient;

FIG. 19B depicts reperfusion recovery half-time in a patient group using a cuff at the ankle and measuring at the foot;

FIG. 19C depicts reperfusion recovery half-time in a patient group using a cuff at the thigh and measuring at the foot;

FIG. 19D depicts reperfusion recovery half-time in a patient group using a cuff at the thigh and measuring at the calf;

FIG. 19E depicts correlation of SNO-Hb level and recovery half-time;

FIG. 20 depicts correlations between NOHb measurements and clinical chemistry results;

FIG. 21 depicts correlations between NOHb measurements and NIRS half-time to hyperemia;

FIG. 22 illustrates an example measurement device;

FIG. 23 is a schematic block diagram illustrating an example system of hardware components capable of implementing examples of the systems and methods disclosed herein.

### DETAILED DESCRIPTION OF SEVERAL EMBODIMENTS

As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

As used herein, an “amount” of a biological substance can represent any of a volume, mass, degree of saturation, or concentration of the substance.

As used herein, a “physiological parameter” is a continuous, categorical, or ordinal value that characterizes a physiological state of a subject.

As used herein, a “predictive model” is a mathematical model that either predicts a future state of a parameter or estimates a current state of a parameter that is not directly measured.

As used herein, a “subject” is a human or other mammal.

As used herein, a “biometric parameter” is a measured parameter that is indicative of the health or fitness of a subject.

As used herein, a measurement is performed “non-invasively” if it is not necessary to remove blood or tissue from a subject to perform the measurement.

As used herein, a “providing therapy to a subject” can include administering a therapeutic, applying mechanical force or electrical energy to the subject, or instructing a subject to perform a specific movement or task believed to provide a therapeutic benefit to the subject.

As used herein, “PNO”, or personal nitric oxide, is an individual's level of the bioactive form of NO in blood that is derived from red blood cells (RBCs) and identified with S-nitrosothiol in hemoglobin, and it is appreciated that SNO in RBCs is in equilibrium with other SNOs, and that PNO may form from different sources of NO (and related NOx) and that S-nitrosothiol release from RBCs may occur in different ways to generate NO and S-nitrosothiols in tissues, and that PNO therefore represents NO bioactivity, including any bioactive form of NO derived from RBCs or otherwise formed in order to oxygenate tissues. It should be understood that PNO is a relative measure representing a direct correlation between oxygen saturation of hemoglobin and total hemoglobin. By way of example, an individual with a higher level of SNO is able to reoxygenate tissue more quickly than an individual with a lower level of SNO, e.g., muscle recovery during exertion such as exercise routines.

As used herein, “UO2” is the amount of oxygen used by the tissue within a region of interest as measured using a nitric oxide based calculation. The calculation is derived as a product of the PNO metric and an amount of oxygen utilized in the tissue.

As used herein, “Max NO power” is a nitric oxide based measure reflecting an individual's maximal rate of energy utilization that relates directly to true measurements of the rate of energy utilization in Watts and is derived from the maximum value of the PNO metric and the maximum energy utilization by the muscle tissue during exercise, and “Max NO endurance” is a nitric oxide based measure reflecting an individual's maximal rate of energy supply that relates directly to increases in critical power, a gold standard measurement of endurance performance, that is derived from a maximum rate of change of the PNO metric generated in a rest period after exercise.

As used herein, a calculation or determination is made in “real time” when it is available to the user within one minute of a corresponding measurement. In one implementation, real time calculations are performed within ten seconds of the measurement.

The amount of oxygen consumed (VO2) by an individual is currently the gold standard measurement of fitness used by physicians and physiologists worldwide. VO2 represents the integrated capacity of the pulmonary, cardiovascular, and muscular system to uptake, transport, and consume oxygen. Traditional systems and methods for measuring VO2 are invasive and/or require carefully controlled conditions. For example, traditional VO2 measurements require the athlete to wear a mask in a lab, and the measurement tools can cost upwards of $35,000.

Embodiments described herein leverage the fact that nitric oxide release during exercise determines how much oxygen is available for muscles to use, and therefore monitor individuals' nitric oxide levels to determine fitness and/or other physiological characteristics. One value that can be measured, referred to as a personalized nitric oxide (PNO), is a measurement of how much active nitric oxide is released from circulating red blood cells during exercise. Active nitric oxide, represented by S-nitrosothiol in blood, opens up blood vessels that deliver oxygen to tissues, including the heart and brain, so a patient's nitric oxide level is strongly linked to their health. By monitoring this metric, and others described below, the system can determine how much nitric oxide an exerciser has released in response to exercise, as well as how intensely an exerciser needs to exercise, how long they should exercise, and what styles of exercise suit them best to provide improvements in fitness, performance, and health. The measured oxygen saturation with the small vessels in muscle is relative to the individual and the context, and thus the PNO metric derived from this measurement is relative across patients and context. It will be appreciated, however, that while the measurement may be relative, it is relative only to a degree, and that an individual's PNO measurement can be used as a reliable indicator of the individual's health and fitness.

The blood volume and oxygen saturation sensors used to calculate UO2 are portable, lightweight, and would less than five percent of many of the devices currently on the market for VO2 measurements. Further, the standard VO2 tests and measurement tools rely on expired gas concentrations to measure systemic oxygen consumption. However, by virtue of taking these central measurements, they miss out on important information about metabolic processes occurring in the muscle. As a result, they cannot reveal why an individual's VO2 maximum is not higher. Because the UO2 measurement is taken at the muscular level (and is influenced by nitric oxide concentrations), it not only measures oxygen consumption, but also allows determination of the rate limiting factors for increasing it (e.g., lack of blood flow or poor muscle use of oxygen). This not only provides a diagnostic measurement tool for fitness, but also indicates what exercise prescriptions are needed to improve health and fitness as well. For example, the origin of a change in UO2 can be determined from a time series of measurements and identified, as either a limitation in either oxygen supply, represented as restricted blood flow, or oxygen utilization, represented as a deficit of muscle function.

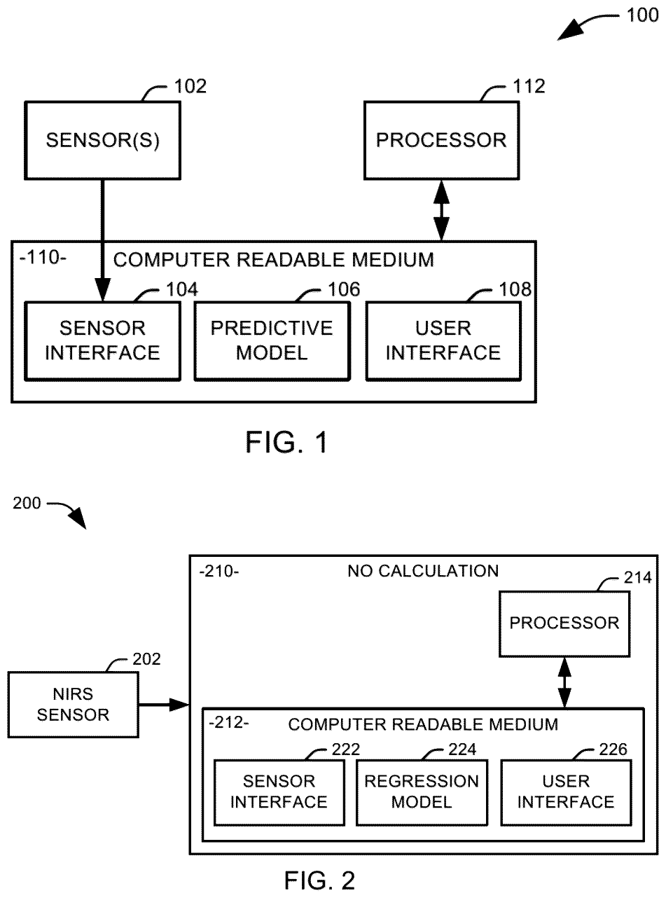

FIG. 1 illustrates an example of a system 100 for generating a value representing an endogenous S-nitrosothiol content of tissue within a region of interest of a subject. It will be appreciated that the system 100 can determine the value representing an endogenous S-nitrosothiol content of tissue both non-invasively and in real-time. This can be used to match the S-nitrosothiol content of the tissue and derived metrics from this content to actions or biometric parameters of the subject during or after exercise or another physiological or external occlusion of blood flow from the muscle tissue. The system 100 may include a set of at least one sensor 102 that non-invasively measures a biometric parameter within the region of interest to provide at least one time series of measurements of the biometric parameter. In one example, where the biometric parameter includes blood volume and oxygen saturation, the set of sensors 102 can include a single sensor that measures both blood volume and oxygen saturation or multiple sensors that collectively provide these measurements. In one implementation, a single optical sensor measures both oxygen saturation and blood volume using near-infrared spectroscopy, determining blood flow according to a change in total hemoglobin concentration and oxygen saturation. It will be appreciated that the set of sensors 102 can include additional sensors that record multiple biometric parameters within the region of interest of the subject generally.

Each of a sensor interface 104, a predictive model 106, and a user interface 108 may be implemented as machine readable instructions stored on a non-transitory computer readable medium 110 and executed by an associated processor 112. The sensor interface 104 may receive the time series of measurements of the biometric parameter from the set of sensors 102 and condition the data for use at the predictive model 104. The predictive model 104 can also utilize data about the subject that is stored at the computer readable medium 110, including, for example, age, sex, genomic data, nutritional information, medication intake, and relevant medical history, as well as any other measured physiological parameters.

The predictive model 110 can utilize one or more pattern recognition algorithms, each of which may analyze the data provided via the sensor interface 104 and any additional data to assign a continuous or categorical parameter to the region of interest representing an amount of endogenous S-nitrosothiol present in the region of interest. Where multiple classification or regression models are used, an arbitration element can be utilized to provide a coherent result from the plurality of models. The training process of a given classifier will vary with its implementation, but training generally involves a statistical aggregation of training data into one or more parameters associated with the output class. For rule-based models, such as decision trees, domain knowledge, for example, as provided by one or more human experts, can be used in place of or to supplement training data in selecting rules for classifying a user using the extracted features. Any of a variety of techniques can be utilized for the classification algorithm, including support vector machines (SVM), regression models, self-organized maps, fuzzy logic systems, data fusion processes, boosting and bagging methods, rule-based systems, or artificial neural networks (ANN).

For example, an SVM classifier can utilize a plurality of functions, referred to as hyperplanes, to conceptually divide boundaries in the N-dimensional feature space, where each of the N dimensions represents one associated feature of the feature vector. The boundaries may define a range of feature values associated with each class. Accordingly, a continuous or categorical output value can be determined for a given input feature vector according to its position in feature space relative to the boundaries. In one implementation, the SVM can be implemented via a kernel method using a linear or non-linear kernel. A trained SVM classifier may converge to a solution where the optimal hyperplanes have a maximized margin to the associated features.

An ANN classifier may include a plurality of nodes having a plurality of interconnections. The values from the feature vector may be provided to a plurality of input nodes. The input nodes may each provide these input values to layers of one or more intermediate nodes. A given intermediate node may receive one or more output values from previous nodes. The received values may be weighted according to a series of weights established during the training of the classifier. An intermediate node may translate its received values into a single output according to a transfer function at the node. For example, the intermediate node can sum the received values and subject the sum to a rectifier function. The output of the ANN can be a continuous or categorical output value. In one example, a final layer of nodes provides the confidence values for the output classes of the ANN, with each node having an associated value representing a confidence for one of the associated output classes of the classifier. The confidence values can be based on a loss function such as a cross-entropy loss function. The loss function can be used to optimize the ANN. In an example, the ANN can be optimized to minimize the loss function.

Many ANN classifiers are fully connected and feedforward. A convolutional neural network, however, includes convolutional layers in which nodes from a previous layer are only connected to a subset of the nodes in the convolutional layer. Recurrent neural networks are a class of neural networks in which connections between nodes form a directed graph along a temporal sequence. Unlike a feedforward network, recurrent neural networks can incorporate feedback from states caused by earlier inputs, such that an output of the recurrent neural network for a given input can be a function of not only the input but one or more previous inputs. As an example, Long Short-Term Memory (LSTM) networks are a modified version of recurrent neural networks, which makes it easier to remember past data in memory.

A rule-based classifier may apply a set of logical rules to the extracted features to select an output class. The rules may be applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees may be trained on random samples of the training set, and an average (e.g., mean, median, or mode) result across the plurality of decision trees is returned. For a classification task, the result from each tree would be categorical, and thus a modal outcome can be used.

The output of the predictive model 106 can be a continuous parameter, representing an amount of endogenous S-nitrosothiol present in the region of interest, or a categorical parameter, representing, for example, an increase or decrease in the amount of endogenous S-nitrosothiol present in the region of interest or classes representing ranges of the amount. The output of the predictive model 106 can be stored, for example, in an electronic health records database and/or provided to a user at an associated display via the user interface 108.

FIG. 2 illustrates another example of a system 200 for non-invasive, real-time generation of a value representing an endogenous S-nitrosothiol content of tissue within a region of interest of a subject. In one example, the tissue is muscle tissue, the endogenous S-nitrosothiol is the endogenous S-nitrosothiol generated from hemoglobin, and the amount of endogenous S-nitrosothiol is determined either during exercise of the muscle or after physiological or external occlusion of blood flow from the muscle tissue. The system 200 may include a near-infrared spectroscopy (NIRS) sensor 202 that non-invasively measures each of a blood volume and an oxygen saturation within the region of interest to provide a time series of blood volume measurements and a time series of oxygen saturation measurements. In one implementation, the time series of blood volume measurements can be represented as a time series for a total hemoglobin metric. A spectroscopy sensor can include a strap such that the sensor is strapped against the skin. The strap can be flexible and/or elastic. As an example, the sensor can be incorporated into a wristband. A person of ordinary skill in the art will recognize that a strap or any other clothing element may be configured to interface a measurement device, as described herein, to any relevant region of interest.

A nitric oxide (NO) calculation assembly 210 may be implemented as machine readable instructions stored on a non-transitory computer readable medium 212 and executed by an associated processor 214. The NO calculation assembly 210 may include a sensor interface 222, a regression model 224, and a user interface 226. The sensor interface 222 may receive the time series of blood volume measurements and the time series of oxygen saturation measurements from the NIRS sensor and condition the data for use at the regression model 224.

The regression model 224 may determine a relationship between the time series of blood volume values and the time series of oxygen saturation values and provide at least one parameter representing the determined relationship. In one example, the relationship is linear, and the ordered pairs provided by the two time series can be fitted to a line of best fit. In this example, the provided parameter is the slope of the line of best fit, with an amount of endogenous S-nitrosothiol in the tissue being derived from the slope. In another example, the parameter is derived from a correlation coefficient between oxygen saturation and blood volume. This value can then be provided to a user via the user interface 226. In one example, instead of or in addition to displaying the value directly, the value can be used for real-time calculation of other metrics representing the health and fitness of the subject.

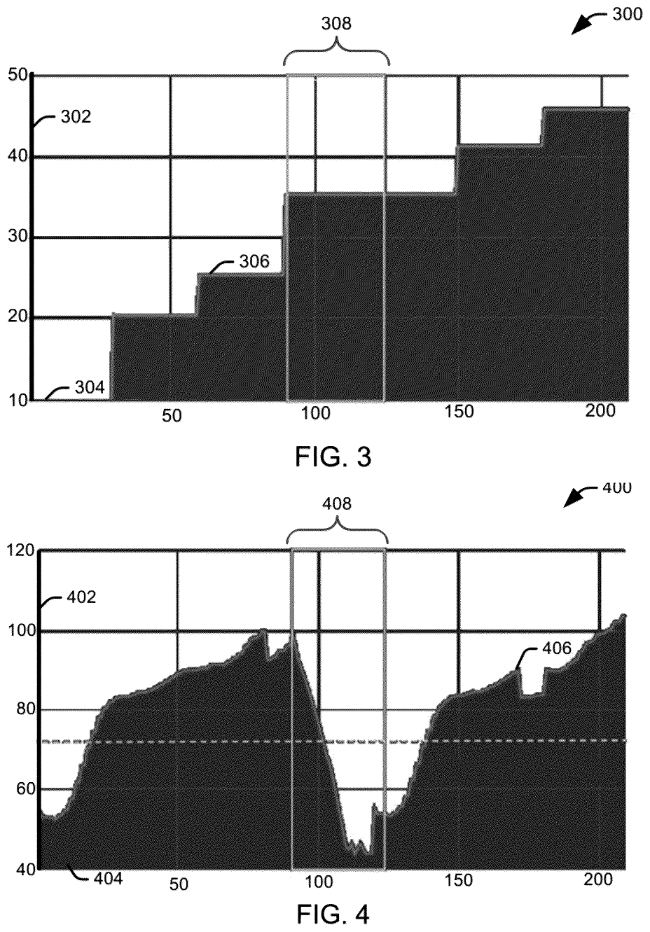

FIG. 3 is a chart 300 illustrating a PNO level for a patient during exercise. The vertical axis 302 represents the PNO level, and the horizontal axis 304 represents a duration of exercise in seconds, with the PNO level at each time indicated as a shaded area 306 on the graph. A rest period 308 was given to the patient around one hundred seconds, and it can be seen that the PNO level 306 stayed level during this period and for a short period thereafter.

As noted above, VO2 is the gold standard measure of fitness used by physicians. Traditionally, VO2 measurement requires invasive testing and expensive lab equipment, but the system 200 allows this measurement to be made non-invasively, within local tissues, making it available during activities of daily living (defined as UO2). FIG. 4 is a chart 400 illustrating a UO2 level for a patient during exercise. The vertical axis 402 represents the UO2 level, and the horizontal axis 404 represents a duration of exercise in seconds, with the UO2 level at each time indicated as a shaded area 406 on the graph. A rest period 408 was given to the patient around one hundred seconds, and it can be seen that the UO2 level 406 fell sharply during the rest period.

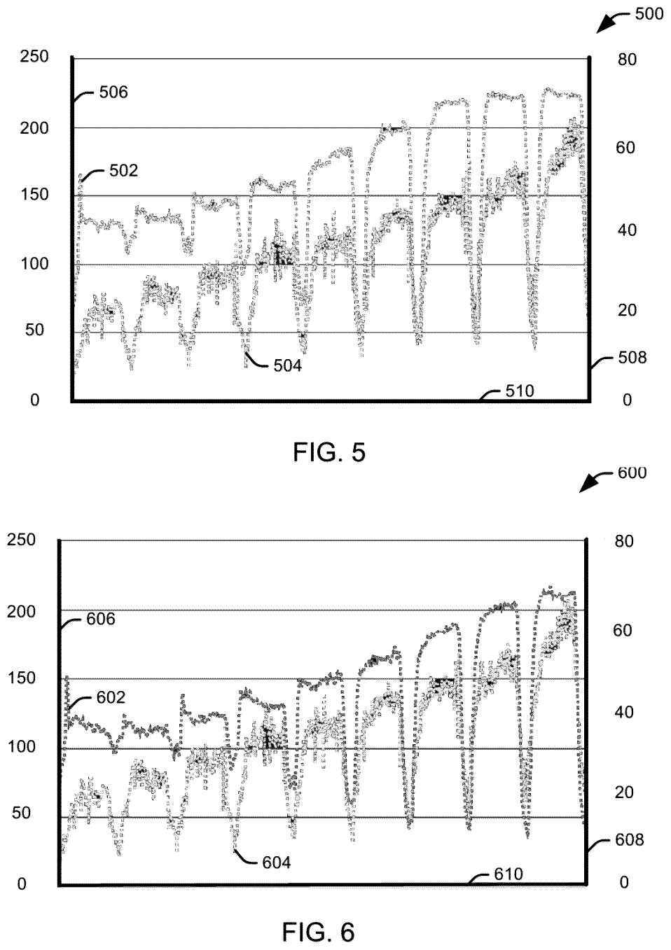

UO2 measurement is a nitric oxide related measure of local muscle oxygen consumption, created to behave in the same way as a true VO2 measure. Specifically, UO2 can be generated as a function of the localized nitric oxide measurement and the blood flow to represent a measure of usable oxygen available for the muscle tissue in a region of interest. As shown in FIGS. 5 and 6, the UO2 measurement is an excellent proxy for VO2, and UO2 can be measured using sensors having a cost of about one-hundredth of the cost for even low-end VO2 measurement devices.

FIG. 5 depicts a chart 500 of a time series of UO2 measurements 502 and a time series of VO2 measurements 504 for an athlete operating a full-body exercise bike using one sensor for recording blood volume and oxygen saturation. The left vertical axis 506 represents VO2 in units of mL/kg/min, the right vertical axis 508 represents UO2, in arbitrary units, and the horizontal axis 506 represents elapsed time. A very strong correlation (r=0.95) can be seen between the measured UO2 502 and the measured VO2 504. From this it should be clear that the estimated VO2 maximum can be deduced from the UO2 measurement. The estimated VO2 maximum in this figure is 68 ml/kg/min, as derived from the UO2.

Similarly, FIG. 6 depicts a chart 600 of a time series of UO2 measurements 602 and a time series of VO2 measurements 604 for an athlete operating a full-body exercise bike using two sensors, on different limbs, for recording blood volume and oxygen saturation. The left vertical axis 606 represents VO2 in units of mL/kg/min, the right vertical axis 608 represents UO2, in arbitrary units, and the horizontal axis 610 represents elapsed time. An extremely strong correlation (r=0.95) can be seen between the measured UO2 602 and the measured VO2 604.

Active nitric oxide levels reflect the supply of oxygen: the better the supply and utilization of oxygen the better the performance. MAX-NO Power and MAX-NO Endurance are nitric oxide related measurements that can be generated by the system that strongly correlate with an individual's real power output and maximal endurance levels.

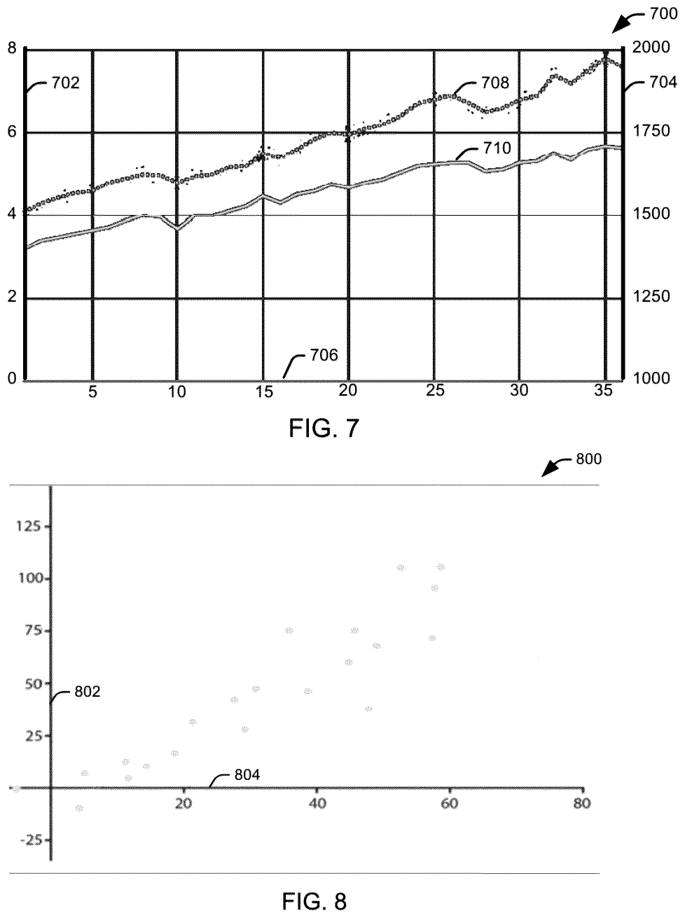

FIG. 7 depicts a chart 700 of an athlete's MAX-NO Power, recorded weekly over a six-month period. The left vertical axis 702 represents the MAX-NO Power in arbitrary units, the right vertical axis 704 represents the maximum power output for the athlete, in watts, and the horizontal axis 706 represents the elapsed time, in weeks. As the athlete increases their fitness, measured by increases in maximal power output 708 in wattage, their MAX-NO Power 710 increases as well. A very strong correlation (R²=0.95) between the measured MAX-NO Power 710 and maximal power output 708 in watts, establishes MAX-NO Power as an excellent biomarker of performance.

FIG. 8 depicts a chart 800 illustrating a relationship between a MAX-NO Endurance improvement for a group of twenty-one athletes and their critical power (a gold standard for endurance, measured in watts), recorded over a six-week period, as a scatterplot. The vertical axis 802 represents a percentage improvement in MAX-NO Endurance for the twenty-one athletes, and the horizontal axis 804 represents the improvement in critical power for the athletes, in watts. As can be seen from the chart, there is a significant correlation between improvement in MAX-NO Endurance and improvement in critical power, establishing MAX-NO Endurance as a non-invasive measure of endurance.

It will be appreciated that specific exercises and other therapies can be prescribed to a patient based upon their values for these metrics. For example, an individual whose Max-NO power is high, compared to their Max-NO endurance, is capable of extracting oxygen from the blood and utilizing it in the skeletal muscle at a greater rate that it can be delivered. These individuals will see the best gains in their exercise regime by performing lower intensity, longer duration, exercise in a continuous fashion. For example, Mr. Jones, a twenty-eight year old with a Max-NO power of 6 and a Max-NO endurance of 3, could be prescribed three days a week of a twenty-minute run at 50-55% of his Maximum UO2. This would be expected to improve his Max-NO Endurance over the course of several weeks.

Alternatively, an individual whose Max-NO endurance is high, compared to their Max-NO power, is capable of supplying oxygen to the working muscles at a much faster rate than they can extract it from the blood and utilize it for energy production. These individuals will see the best gains in their exercise regime by performing high intensity, short duration, work bouts with rest periods interspersed between them. In these instances, there will be very large acute increases in UO2 with relative low points between exercise bouts. For example, Mrs. Benneton, a forty-eight year old cyclist with a Max-NO endurance of 7 and a Max-NO power of 2.5, could be prescribed two days a week of sprinting at a near maximal intensity until her UO2 stops rising then resting for three minutes, repeating for six sets. This would be expected to improve both her Max-NO Power and max UO2 over the course of several weeks.

In another example, an individual whose upper body PNO levels are high compared to their lower body PNO levels will be instructed to redistribute their power output such that they decrease the amount of work their upper body is doing and increase the amount of work their lower body is doing. By doing so they will increase PNO levels in their lower body, leading to a greater full body PNO value which can be sustained for a longer duration. This will increase the delivery of oxygen to the brain, heart, and muscles. In another example, an individual suffering from early onset Alzheimer's Disease can be assigned exercises to improve their PNO level, and thus improve blood flow to the brain. For example, Mrs. Levy, a seventy-year-old with early onset Alzheimer's disease could be prescribed daily exercise of thirty minutes of walking with the goal of increasing her PNO of 5. Mrs. Levy could be prescribed a thirty-minute daily bicycle routine that is titrated such that her PNO increases to 15 over the course of the workout. At six months, improvement of both her memory and her baseline PNO would be expected.

As discussed, VO2 is currently the gold standard measurement of fitness used by physicians and physiologists worldwide and represents the integrated capacity of the pulmonary, cardiovascular, and muscular system to uptake, transport, and consume oxygen. UO2 measurement is a nitric oxide related measure of local muscle oxygen consumption, created to behave in the same way as a true VO2 measure. Specifically, UO2 can be generated as a function of the localized nitric oxide measurement and the blood flow to represent a measure of usable oxygen available for the muscle tissue in a region of interest. As shown in FIGS. 5 and 6, the UO2 measurement is an excellent proxy for VO2.

Individuals with Alzheimer's Disease are known to have low VO2. VO2 largely depends on microvascular blood flow and nitric oxide from red blood cells controls blood flow. Thus, it stands to reason that the nitric oxide-based measurement described herein as UO2, tracks VO2 and therefore PNO can be used to predict VO2 max in an individual. Therefore, in another embodiment, the invention provides a method of determining a risk of Alzheimer's Disease or early onset disease by using UO2 as a biomarker. If one improves their UO2 (and PNO), there would be an improvement or protection against the Alzheimer's Disease. In one aspect, the invention provides a method of determining a risk for diseases that associate with reduced blood flow, for example, dementia or other reductions in cognitive function associated with blood flow or cardiovascular/cardiometabolic disease. An individual with Alzheimer's Disease, for example, is prescribed an exercise regimen and their UO2 measurements are taken over time to determine improvement in the disease. For example, UO2 is measured at a beginning time point before an exercise regimen is commenced and measured at a second time point (and optionally further time points) to determine if there is an increase in the UO2 value, reflecting an improvement of cognitive function or dementia, for example. Other complementary tests can be used including cognitive tests known to those of skill in the art to further assess improvement in the disease state of the individual.

In another example, values representing an endogenous S-nitrosothiol content of tissue, such as UO2 measurements, can be used to determine the presence of or risk of a neurodegenerative disorder, such as Parkinson's Disease. For example, a low UO2 measurement relative to a subject not having the neurodegenerative disorder is indicative of a risk of the neurodegenerative disorder. For example, the systems and methods disclosed herein can be used to monitor the NO and/or sNO of the patient and compare it to a standard or personalized threshold associated with maintaining the function of the blood/brain barrier in a state to provide the proper amount of 0, blood nutrients, and trophic factors, such as Brain-derived neurotrophic factor (BDNF), to keep brain cells functioning properly. Parkinson's Disease, in particular, has been shown to respond to intense exercise, which impacts sNO levels. See, e.g, Salgado, Sanjay, Nori Williams, Rima Kotian, and Miran Salgado. 2013. “An Evidence-Based Exercise Regimen for Patients with Mild to Moderate Parkinson's Disease” Brain Siences 3, no. 1: 87-100. https://doi.org/10.3390/brainsci3010087; Maggie Fox (Dec. 11, 2017). “Vigorous exercise can slow Parkinson's” NBC News. https://www.nbcnews.com/health/health-news/vigorous-exercise-can-slow-parkinson-s-n828521; and Schenkman, Margaret et al. “Effect of High-Intensity Treadmill Exercise on Motor Symptoms in Patients With De Novo Parkinson Disease: A Phase 2 Randomized Clinical Trial.”. JAMA neurology vol. 75,2 (2018): 219-226. doi: 10.1001/jamaneurol.2017.3517. Each of these references are hereby incorporated by reference.

In another example, Mr. Jack is a sixty-year-old businessman with heart disease. He could be prescribed a daily exercise regime of thirty minutes to improve his PNO level of 13. In this example, his exercise regime could be increased to forty minutes over time with a doubling of PNO, representing an increased ability to provide oxygenated blood to the heart muscle. In another example, Mrs. Stevenson is a sedentary mother with three young children who she struggles to keep up with in daily living with a Maximum UO2 of 43. She could be prescribed an exercise regime consisting of two days per week, with the first day including twenty to thirty minutes of moderate intensity exercise at 50-60% of her Maximum UO2 and the second day including three five-minute exercise bouts at 75-85% of her Maximum UO2. This would be expected to increase her fitness and energy, as well as her maximum UO2. Likewise, in another example, Mr. James is a sixty-year-old businessman with diabetes. His baseline blood sugar was 200. He could be prescribed a daily exercise regime of 20 minutes to improve his PNO level of 12. In this example, his exercise regime could be increased to forty minutes over time with a doubling of PNO, representing an increased ability to provide oxygenated blood to muscles and reduce his resting blood sugars.

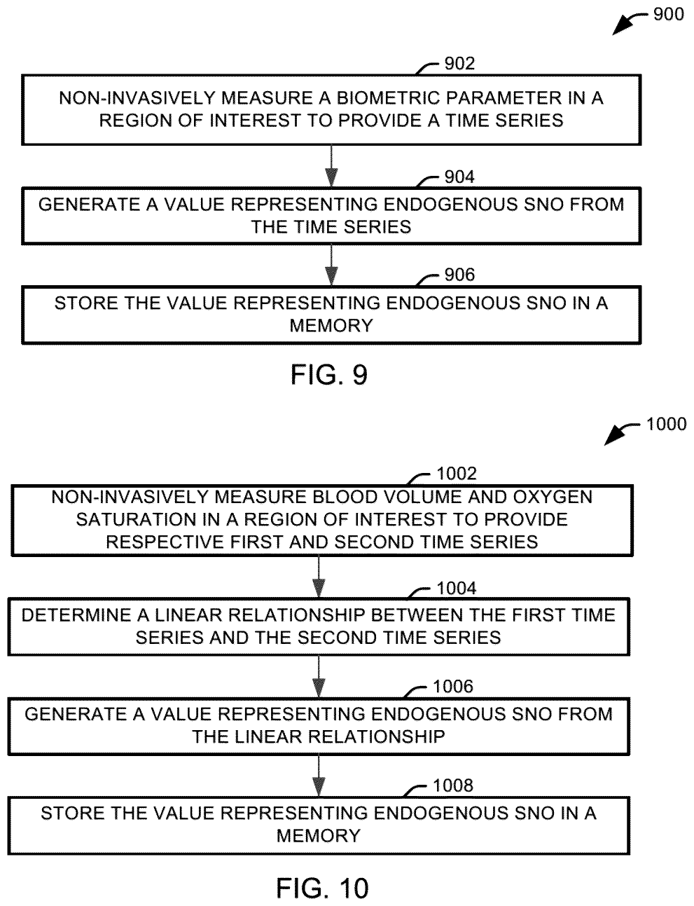

In view of the foregoing structural and functional features described above, example methods will be better appreciated with reference to FIGS. 9-11. While, for purposes of simplicity of explanation, the example methods of FIGS. 9-11 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement a method. Each of these methods can be performed by system 100 of FIG. 1 and/or system 200 of FIG. 2, for example.

FIG. 9 illustrates one example of a method 900 for generating a value representing an endogenous S-nitrosothiol content of tissue within a region of interest of a subject. At 902, a biometric parameter may be measured non-invasively by sensor(s) 102/202 within a region of interest of a subject to provide a time series of measurements of the biometric parameter. In one example, these measurements are taken while the subject is engaging in exercise. In another example, these measurements are taken during a rest period after the subject has engaged in exercise. In a further example, the measurements can be taken immediately after a physiological or external occlusion of blood flow to the region of interest or after physiological depletion of oxygen. In one implementation of this example, an overshoot response in one of blood flow and oxygen saturation above baseline following an induced hypoxia is measured to provide one of the time series of oxygen saturation measurements and the time-series of blood volume measurements. The predictive model uses the overshoot or rate value to generate a value representing an endogenous S-nitrosothiol content of tissue within the region of interest.

At 904, a value representing an endogenous S-nitrosothiol content of tissue within the region of interest may be generated by processor 112/214 from the time series via a predictive model 106/224. In one implementation, a linear relationship between a first time series, representing blood volume, and a second time series, representing oxygen saturation, is determined, and the value is determined according to this linear relationship. For example, the two time series can be provided to a linear regression model to provide a best-fit line between the oxygen saturation and the blood volume over time, with the value representing the endogenous S-nitrosothiol content of tissue within the region of interest being derived from a slope of the best-fit line. At 906, the value representing an endogenous S-nitrosothiol content of tissue within the region of interest may be stored by processor 112/214 in a memory implemented as a non-transitory computer readable medium 110/212. In one example, the method 900 can be performed before and after a therapy provided to the subject to determine an effect of the therapy on the endogenous S-nitrosothiol content within the region of interest by comparing a value generated after the therapy compared to a stored value generated before the therapy. The stored value can also be used to generate one or more of a maximum nitric oxide endurance metric, a maximum nitric oxide power metric, a usable oxygen consumption metric, and a personalized nitric oxide metric for the subject.

FIG. 10 illustrates another example of a method for generating a value representing an endogenous S-nitrosothiol content of tissue within a region of interest of a subject. At 1002, blood volume and oxygen saturation within a region of interest of a subject may be measured by sensor(s) 102/202 non-invasively to provide a first time series of oxygen saturation measurements and a second time series of blood volume measurements. For example, both blood volume and oxygen saturation in the tissue can be determined via near-infrared spectroscopy. In one example, these measurements are taken while the subject is engaging in exercise. At 1004, a linear relationship between the first time series and the second time series may be determined by processor 112/214 via a predictive model 106/224. For example, the two time series can be provided to a linear regression model to provide a best-fit line between the oxygen saturation and the blood volume over time.

At 1006, a value representing an endogenous S-nitrosothiol content of tissue within the region of interest may be generated by processor 112/214 from the linear relationship between the first time series and the second time series. In one implementation, in which the linear relationship is represented as a best-fit line between the first time series and the second time series, the value representing the endogenous S-nitrosothiol content of tissue within the region of interest can be derived from a slope of the best-fit line. At 1008, the value representing an endogenous S-nitrosothiol content of tissue within the region of interest may be stored by processor 112/214 in a memory implemented as a non-transitory computer readable medium 110/212. The stored value can also be used to generate one or more of a maximum nitric oxide endurance metric, a maximum nitric oxide power metric, a usable oxygen consumption metric, and a personalized nitric oxide metric for the subject.

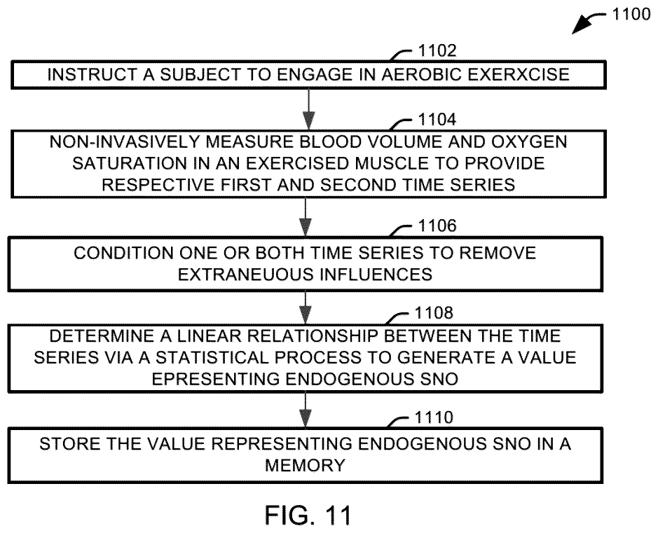

FIG. 11 illustrates a further method 1100 for generating a value representing an endogenous S-nitrosothiol content of tissue within a region of interest of a subject. At 1102, a subject is instructed to engage in aerobic exercise. In one example, a subject could be instructed to cycle an exercise bike. At 1104, blood volume and oxygen saturation within a region of interest of a muscle of the subject that is impacted by the exercise may be measured non-invasively by sensor(s) 102/202 to provide a first time series of oxygen saturation measurements and a second time series of blood volume measurements.

At 1106, at least one of the time series of blood volume and the time series of oxygen saturation within a region of interest may be conditioned by processor 112/214 to remove extraneous influences. Blood flow in tissues is mediated by many different factors, including prostaglandins, catecholamines, nitric oxide, temperature, kinins, adenosine triphosphate (ATP), hypoxia, and similar factors. For example, kinins regulate flow increases during inflammation and NO mediates shear and Ach induced vasodilation. To facilitate measurement of the effects of NO released from hemoglobin on blood flow, the collected blood flow data can be conditioned to remove the effects of these factors. For example, most aerobic exercise involves repeated contraction of the muscle on a somewhat predictable period. This reduction of blood volume can be quantified as a periodic signal, represented as another time series, which can be removed from the time series of blood volume measurements. Other physiological influences on one or both of the blood volume and the oxygen saturation will vary with the location and the specific exercise, and these non-linear influences on the relationship between blood volume and oxygen saturation can be identified and removed from the time series by adding signals representing these influences. At 1108, a statistical process may be performed by processor 112/214 to identify a linear relationship between the blood flow, as derived from the time series of blood volume measurements, and the oxygen saturation, and a value representing an endogenous S-nitrosothiol content of tissue within the region of interest is generated from the linear relationship. For example, a linear regression analysis can be performed, and the slope of a best-fit line generated in the regression analysis can be used to quantify the linear relationship between the blood flow and the oxygen saturation. A correlation coefficient between the two parameters can also be generated and used to evaluate the linear relationship where the muscle is particularly depleted of oxygen and the slope cannot be readily established. At 1110, the value representing an endogenous S-nitrosothiol content of tissue within the region of interest may be stored by processor 112/214 in a memory implemented as a non-transitory computer readable medium 110/212.

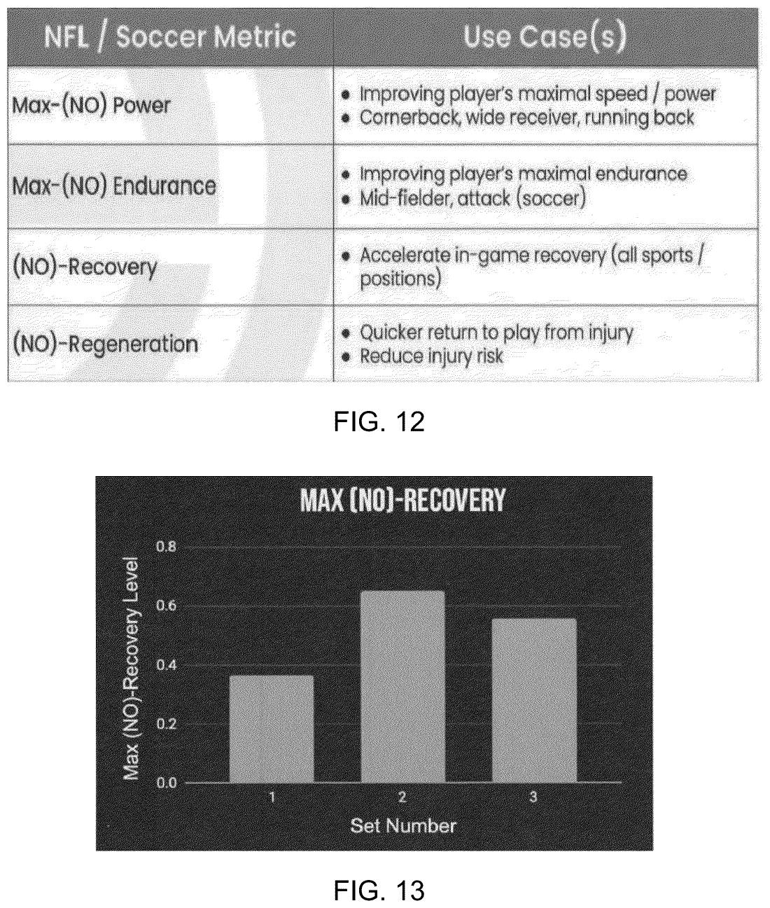

FIG. 12 illustrates metrics and use cases for professional athletes. Specific metrics can correlate to different positive athletic outcomes such as the improvements in maximal speed, power, or endurance. Additionally, metrics may aid in monitoring, predicting, and planning an athlete's recovery and regeneration. For example, measurements of Nitric Oxide can be used to assist in developing exercise activities that cause the maximum endogenous increase in Nitric Oxide in a person's blood supply. In another example, Nitric Oxide recovery can be monitored to determine how much rest the athlete needs in-game or between sets. In a third example, Nitric Oxide regeneration can be monitored and/or improved through targeted exercise to reduce injury risk and expedite return to play from injury.

FIG. 13 depicts an example measurement of MAX NO Recovery versus the number of sets performed in exercise routine. Max (NO)-Recovery is derived from the rate of reoxygenation in a muscle tissue following an exercise bout, and it is influenced by various factors such as (NO) concentrations, breathing patterns, and aerobic fitness levels. Max (NO)-recovery can tell an athlete how recovered they are in live time, as well as when they are fully recovered after an exercise bout. Additionally, individuals can train to increase their Max (NO)-Recovery scores, which will allow them to recover quicker after or between exercise bouts. As noted in the chart, the athlete's MAX NO Recovery peaks at two sets. A threshold can be set for a drop from the peak MAX NO Recovery to determine a maximum amount of sets the athlete should perform. Through conditioning, an athlete may achieve peak at a greater number of sets and/or the drop rate after the peak value may lessen.

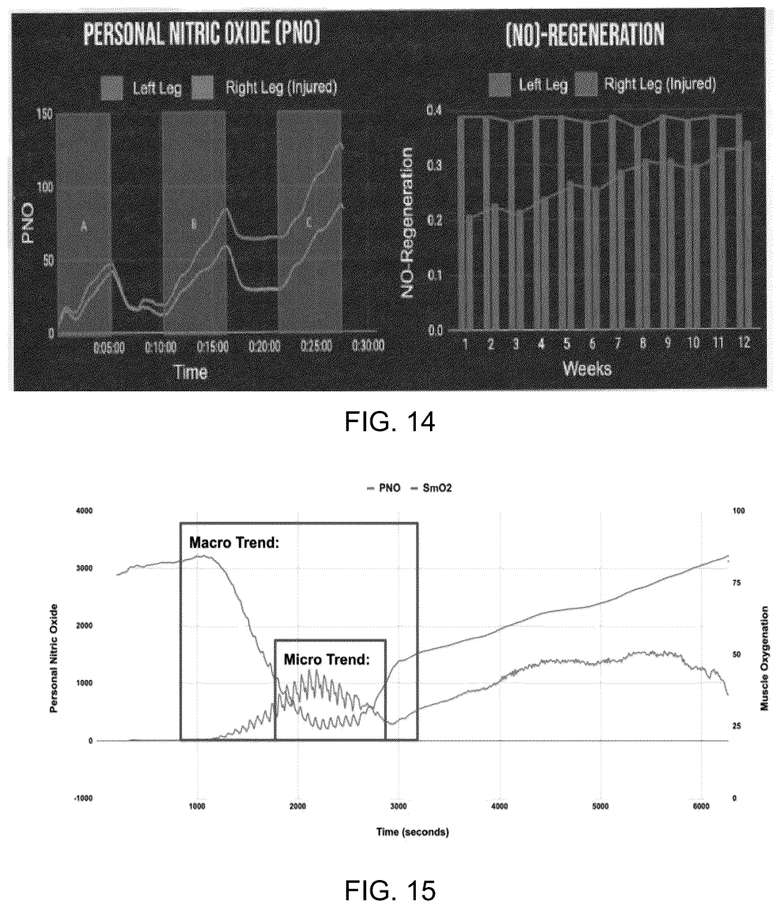

FIG. 14 depicts an example measurement of PNO and MAX NO Regeneration over time. The PNO measurement charts an athlete during an exercise session through alternating periods of work and rest. While the athlete exercises, PNO increases. During a recovery period, PNO drops back down. The more PNO the athlete generates, the better their health and performance. PNO can also be used to mitigate injury risk and decrease an individual's risk of reinjury during rehabilitation. PNO levels can be compared between a healthy and injured leg which informs the athlete about their ability to handle loading.

By comparing an injured person's level of Nitric Oxide taken at different times, one may determine the level of tissue injury recovery that the injury repair has made relative to a total recovery. The measurement for MAX NO Regeneration can be calculated at rest. To record Max NO Regeneration a cuff can be placed on an individual's upper arm or upper thigh, then the biosensor can be placed on a large muscle distal to the cuff The cuff can be inflated until the pressure occludes blood flow to the limb. Once blood flow is occluded the individual can remain stationary with the cuff inflated for a fixed time period, after which the cuff may automatically deflate, allowing blood to flow back into the limb. Biomarker measurements can be recorded during the post ischemia reperfusion period and can be used to calculate NO levels.

In the example measurement, the athlete's MAX NO regeneration scores are depicted on their left and right leg for 12 weeks after receiving right ACL surgery. The difference between the healthy left leg and injured right leg are easily trackable throughout the recovery process. The metric can be used in combination with existing metrics, such as strength. The metric can be used to optimize a player's training regimen to get them back on field as quickly as possible without undue risk of injury.

As seen in FIG. 15, there are separate trends occurring simultaneously, when measuring PNO or muscle oxygenation (SmO2). The macro trend represents the auto-regulation of blood flow. When oxygen is utilized in the skeletal muscle there is a compensatory increase in muscle blood flow. When this occurs, an inverse linear correlation can be observed between muscle oxygenation and total hemoglobin (THb), which is a measure of muscle blood volume (not depicted). As a result, a gross increase in nitric oxide and s-nitrosothiols (PNO) can be observed. This response can last seconds to minutes.

The micro trend represents active hyperemia. When a muscle is contracted, blood flow is restricted, and as a result, oxygen levels decline. Then, during the muscles relaxation phase before the next contraction, blood flow and oxygen saturation increase. During active hyperemia, SmO2 and THb are linearly correlated. The active hyperemic response is depicted in the micro-trend in FIG. 15 as the rapid increases and decreases in PNO.

A person of ordinary skill in the art will note that the macro trend is the gross increase in PNO during exercise, but within the macro trend there is a micro trend consisting of smaller increases/decreases in PNO. Together, these two trends work to regulate muscle blood flow.

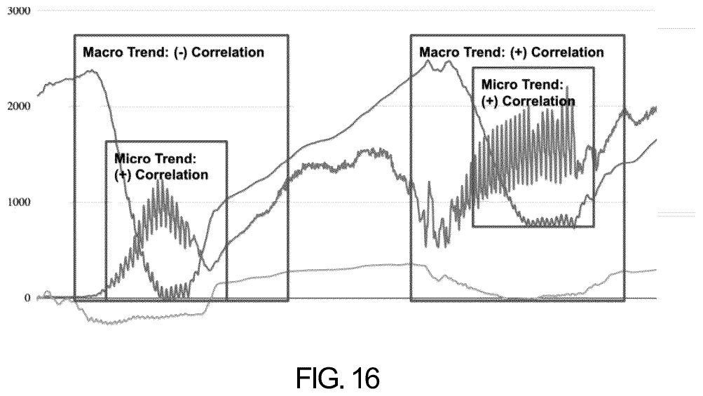

As seen in FIG. 16, there are also cases where the macro trend and micro trend show positive linear correlations, as would be the case when muscle contraction are having a stronger than usual impact on blood flow. As a result, the auto-regulatory response cannot be clearly observed. However, because linear correlations, whether positive or negative, are being examined, the PNO still increases.

In FIG. 16, there is a (+) correlation nested within a (−) correlation as well as a (+) correlation nested within a (+) correlation. While it is not possible to visually distinguish between the two, the possibility is worth mentioning (the second set has higher PNO for reasons other than the fact that it is a +/+ correlation).

Following a tissue injury, an injured person can believe the tissue injury has fully recovered, and full weight can be placed on the injured tissue and/or the injured body part is fully functional when in fact this tissue injury has not yet fully recovered. The determination for actual full tissue injury recovery can be made by comparing the Nitric Oxide levels from the injured limb and the opposing, uninjured limb of the same type (i.e., arms or legs). Based on the metrics described herein, the injured person can proceed with full activity with a reduced possibility of reinjury.

### Example: Study

To establish the importance of SNOs, specifically SNO-Hb-βCys93, in clinically relevant measures of hypoxic vasodilation, mice expressing human Hb were utilized. The mice are depleted in SNO-Hb and show a number of cardiovascular deficits resulting from impairment of hypoxic vasodilation. In the study, the functional consequences of Cys93 SNO emulating a standard clinical protocol for reactive hyperemia: reoxygenation in gastrocnemius muscle (as measured by pO2 with a needle electrode) after five minutes of femoral artery occlusion was examined.

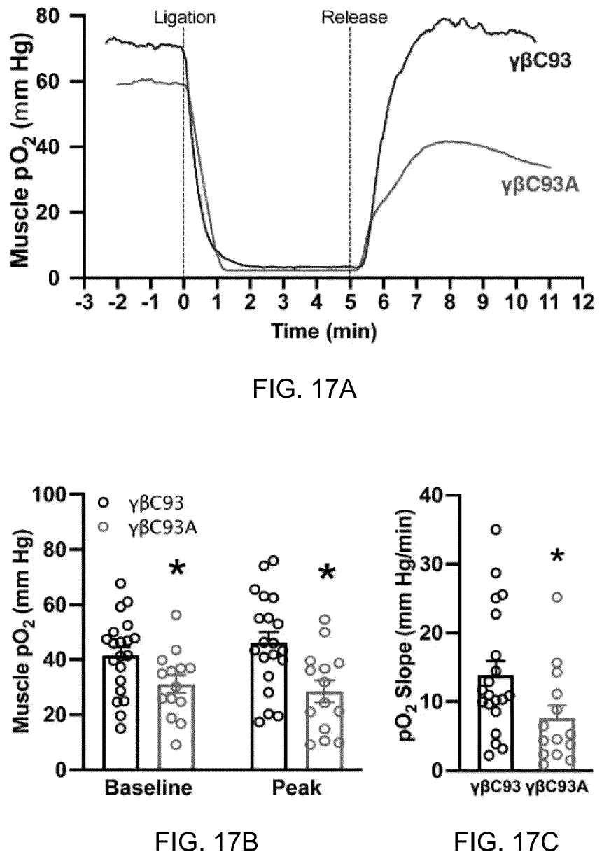

FIG. 17A depicts representative tracings from a control βOC93 mouse and a corresponding βOC93A mutant animal. In the βOC93 mouse with normal hypoxic vasodilatory activity, the tissue pO₂ response following release of the artery occlusion (i.e., restoration of femoral artery flow) was a rapid recovery that even overshot the baseline. In contrast, the βOC93A animal exhibited a delayed response and muscle oxygenation did not return to baseline during the five-minute post-release recording interval. Group data comparisons showed that basal pO₂ in gastrocnemius muscle was significantly lower in the βOC93A mice, as shown in FIG. 17B; p=0.032, consistent with our previous study. The group data comparisons additionally showed that the recovery of muscle pO₂ after five minutes in βOC93A mice was blunted compared to βOC93 controls (46±17 vs. 28±15 mm Hg; p=0.004). The group data comparisons also showed that the rate of post-occlusion recovery in muscle pO2 was significantly reduced compared to that of γβC93 mice, at about half of the normal rate (0.23±0.15 vs. 0.13±0.11 mm Hg/sec, respectively), as shown in FIG. 17C; p=0.036. Thus SNO-Hb deficiency reduces the rate and overall efficiency of tissue oxygenation resulting from a brief interruption in blood flow.

RBC SNO levels were measured in age-matched healthy controls versus patients diagnosed with diseases characterized by systemic (i.e., heart failure and chronic obstructive pulmonary disease (COPD)) or peripheral (i.e., peripheral vascular disease and sickle cell disease) deficiencies in oxygenation: diabetic peripheral artery disease (PAD); heart failure with reduced ejection fraction (HF); COPD; and sickle cell disease (SCD). Specific inclusion criteria and disease status for each cohort are provided in the extended methods. 53 subjects, with 49 individuals completing the study (13 healthy, 13 PAD, 6 HF, 9 COPD, and 8 SCD), were enrolled.

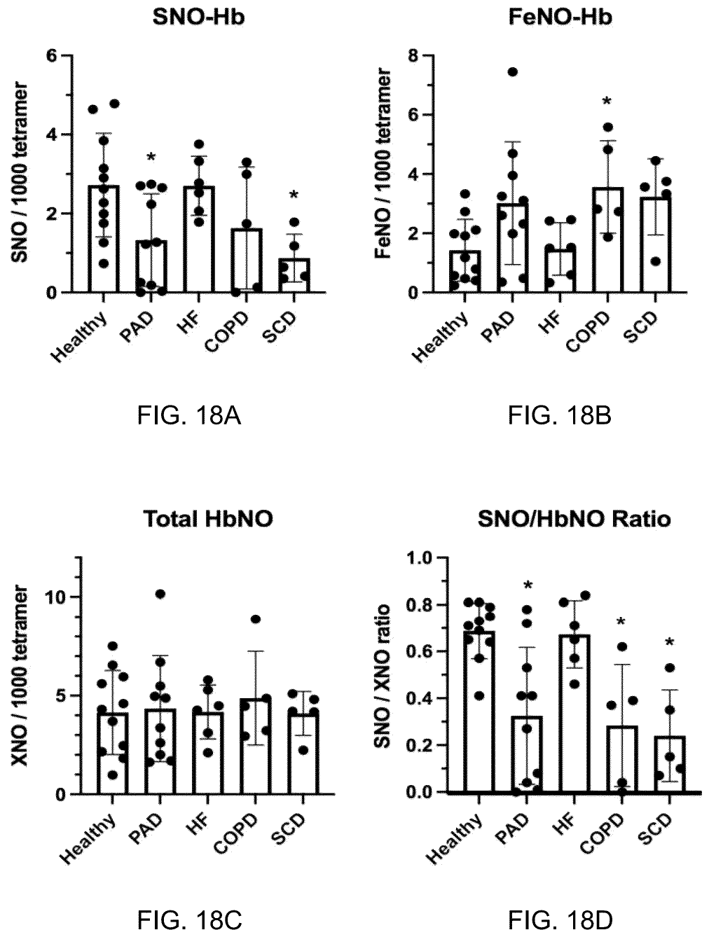

RBCs were processed on site and SNO-Hb and iron-nitrosyl hb was quantified by Hg coupled photolysis-chemiluminescence within ˜1 hour of procurement from the radial artery. Values from nine subjects were discarded due to an instrument malfunction (i.e., the diagnosis and the decision to discard those data were made by technical staff unaware of the patients' physiologic status). Results from the remaining samples are presented in FIGS. 18A-D.

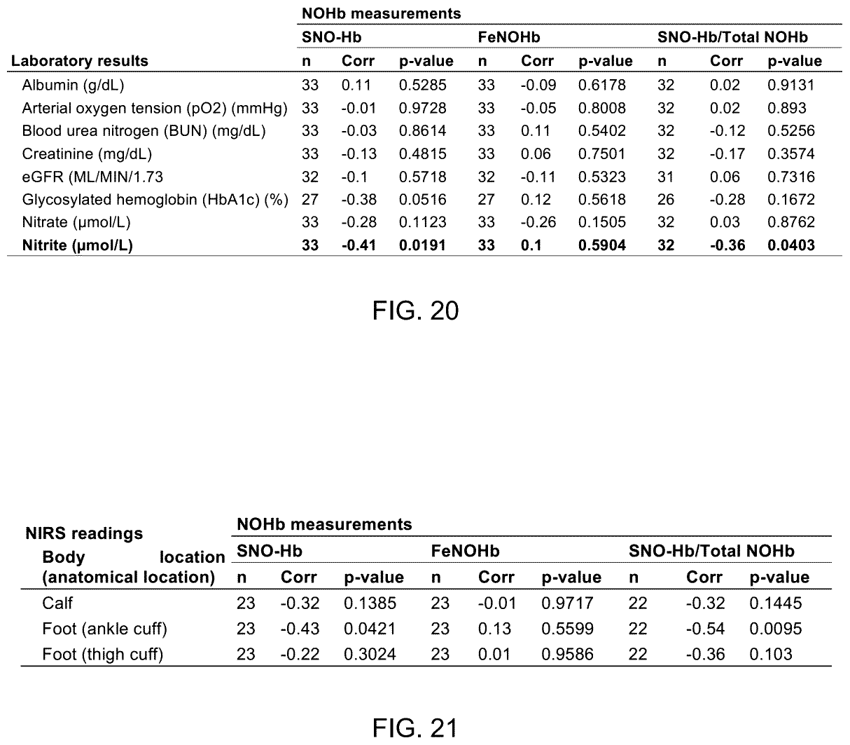

In the normal volunteers (n=10), arterial RBC SNO-Hb levels were 2.6±1.3 per 1000 Hb, as shown in FIG. 18A, a concentration similar to what was recorded in other groups of healthy subjects. SNO-Hb level in the HF cohort (n=5; 2.5±0.6 per 1000 Hb) and COPD cohort (n=5; 2.0±1.3) did not differ from controls. However, the amount of SNO-Hb in the blood from the PAD (n=11; 1.5±1.2) and SCD (n=5; 0.9±0.6) patients were significantly lower than in the normal controls (p<0.05). Levels of SNO-Hb can decline because of overall decreases in NO production or because of processing defects within the Hb molecule that prevent intramolecular transfer of NO from heme to thiol, as previously reported for SCD, PH, and for healthy subjects under hypoxia, reflected in increases in amounts of inactive FeNO. Notably, the total amount of NO bound to Hb (HbNO) did not differ from normal in any patient group, as shown in FIG. 18C. However, HbFeNO concentrations, as shown in FIG. 18B, were significantly higher than normal in the PAD, COPD, and SCD patient groups, reflecting a significant decline in the ratio of SNO to total HbNO in all groups except HF: from 0.69±0.13 and 0.67±0.16 in the normal volunteers and HF patients, respectively, to 0.36±0.30 in PAD, 0.36±0.22 in COPD, and 0.24±0.20 in SCD. Exploratory analysis of correlations between SNO-Hb and various clinical chemistry parameters was also conducted. FIG. 18C depicts total HbNO and FIG. 18D depicts a ratio of SNO to total HbNO for the identified patient group. Analyses of the data set, as shown in FIG. 20 (n=33), identified inverse correlations between plasma nitrite levels vs. SNO-Hb, and nitrite vs. the SNO-Hb/total HbNO ratio. Differences in SNO-Hb and FeNO levels, the ratio of SNO to total HbNO, and negative correlations between plasma nitrite and NO bioactivity are all suggestive of NO processing defects in the RBCs from PAD, COPD, and SCD patients.

The study sought to confirm the role of SNO-Hb in reactive hyperemia as demonstrated in βOC93A mice. Following blood procurement, calf and foot tissue oxygenation were measured using a near-infrared spectroscopy (NIRS) device following brief periods of limb blood flow occlusion. Patients with SCD were excluded from this arm of the study due to the potential for leg ischemia to induce a vaso-occlusive crisis. Subjects in the other cohorts were placed semi-supine with inflatable cuffs wrapped around their upper thigh and lower calf near the ankle. Each cuff was rapidly inflated over one second to halt arterial blood flow (target pressure=systolic blood pressure+˜130 mm Hg; max 300 mm Hg), and the occlusive period held for five minutes, before releasing the cuff pressure and measuring tissue oxygenation for five minutes. There was a five-minute recovery interval between the two-cuff inflation/recording sessions, with the ankle cuff being used first with recording at the foot, and the thigh cuff being used second, with separate recordings at both foot and upper calf. Occlusion testing was conducted on 45 subjects, with the NIRS tracings analyzed off-line by individuals unaware of the subjects' disease status or RBC SNO levels. Thirteen of the resultant NIRS recordings were deemed uninterpretable by the independent analysis group due to leg motion and/or poor signal resolution and thus excluded, leaving data from 11 healthy, 8 PAD, 6 HF, and 7 COPD patients for comparative purposes.

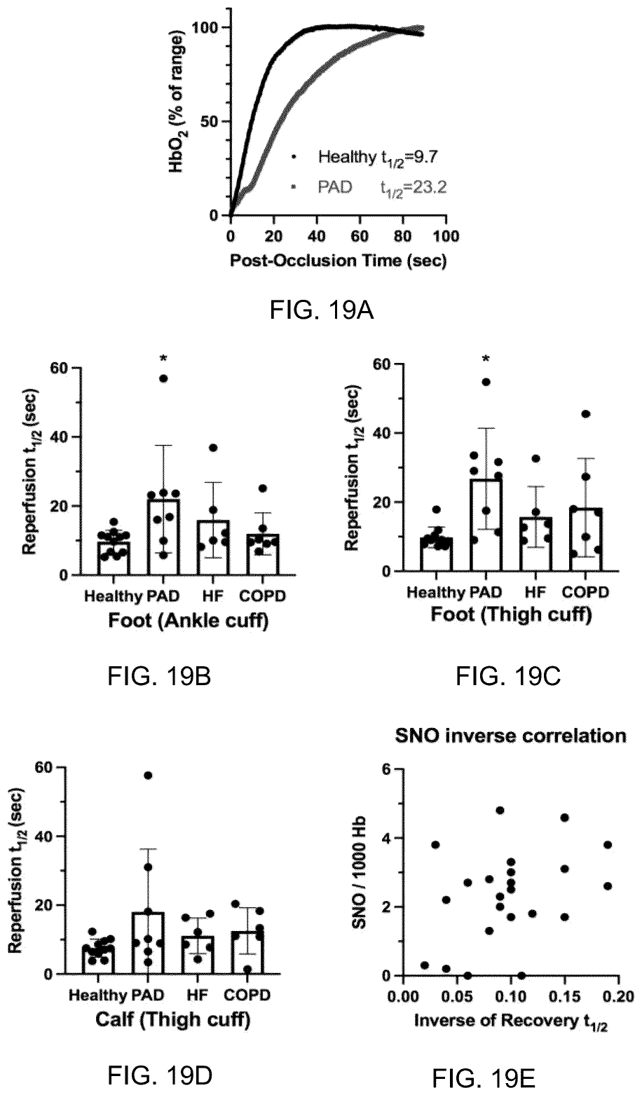

The experimental endpoint was half-time measured in seconds (t_{1/2}) to restoration of tissue oxygenation (i.e., 50% return to baseline) and the findings are presented in FIGS. 19A-E. Representative foot tissue oxygenation recovery tracings from one healthy subject and one PAD patient are shown in FIG. 19A after release of the thigh cuff. The healthy subject had a robust and rapid re-oxygenation response with t_{1/2} of 10 sec, while the PAD patient exhibited a delayed tissue oxygenation response with a t_{1/2} of 22 seconds. This is very similar to the rapid restoration of tissue pO₂ in the βOC93 control mice following release of femoral artery occlusion versus slower recovery in βOC93A mice, as shown in FIG. 17A. Quantified group data (mean±SD) showing the t_{1/2} for foot re-oxygenation following ankle and thigh cuff occlusion and for calf re-oxygenation following thigh occlusion are presented in FIGS. 19B, 19C, and 19D, respectively. For all three measures, the healthy subjects recorded mean t_{1/2} values of approximately 10 seconds, consistent with previous studies. Importantly, as a group there was a direct correlation between SNO-Hb levels and reperfusion rate, as shown in FIG. 19E. Numerically higher mean t_{1/2} values were observed for all three patient groups, but the foot reperfusion half-times following ankle or cuff inflation significantly increased only in the PAD subject group. Further, there was a significant inverse correlation between foot re-perfusion t_{1/2} vs. SNO-Hb levels and vs. the SNO-Hb to total HbNO ratio but not vs. FeNO levels, as shown in FIG. 21, thus linking RBC SNO to reoxygenation response.

Microvascular blood flow is impaired in many clinical conditions resulting in tissue ischemia. However, drugs that increase blood flow do not improve tissue oxygenation. Also, clinical measures of blood flow have focused on the endothelial component, in particular NO, which plays no role in tissue oxygenation. On the other hand, there is strong evidence that blood flow governing tissue oxygenation is regulated by S-nitrosohemoglobin. Alternatively stated, blood flow subserving blood pressure is regulated by endothelial NO whereas blood flow regulating tissue oxygenation is controlled by RBC-SNO. Reactive hyperemia is the increase in blood flow following transient ischemia that occurs to restore tissue oxygenation. Whereas reactive hyperemia has been attributed to endothelial NO, RBC-SNO has a major role in both vasodilatory and blood flow responses in mice. The study expands that work to include direct measure of tissue oxygenation in both mice and humans. Importantly, the study demonstrates that SNO-Hb is required to oxygenate hypoxic tissues and that deficits in SNO-Hb lead to impairments in oxygenation. Moreover, levels of SNO-Hb in patients predict tissue oxygenation following a brief period of localized hypoxia indicating a first biomarker of microcirculatory blood flow.

The study presents a model of a 3-gas model for the respiratory cycle where O2/NO are loaded on to Hb simultaneously and SNO-Hb then releases vasodilatory SNO to adjust blood flow with tissue oxygen delivery. In the mutant mice unable to carry or dispense SNO from βCys93, tissue oxygenation is therefore broadly impaired. In addition to tissue hypoxia under basal conditions, mutant mice exhibit deficits in tissue oxygenation under global hypoxia and under regional ischemia, as shown as shown in FIG. 17B. Conversely, hypoxic conditions that impair oxygen loading, or otherwise impair the allosteric transition in Hb, also impair S-nitrosylation. This manifests either in terms of lower SNO-Hb levels or lower ratio of SNO-Hb to total HbNO, since NO still binds heme iron, only it cannot transfer to Cys93. Correspondingly, patients with disease states characterized by pathologies of oxygen (COPD, PAD, and SCD) exhibited lower levels of SNO-Hb and lower SNO/HbNO, as shown in FIGS. 18A-D, confirming previous reports. Predictably, the ratio of SNO-Hb to total Hb-associated NO (SNO-Hb plus Hb FeNO; total HbNO), indicative of accumulation of inactive FeNO, was a more sensitive measure of loss of bioactivity than measures of SNO-Hb alone. Thus, NO/SNO processing defects are observed in multiple diseases characterized by deficient tissue oxygenation and may be causally linked by a shared inability of Hb to convert FeNO to SNO-βCys93-Hb. By the same token, we found that the ratios of SNO-Hb to nitrite and of SNO-Hb/HbNO to blood nitrite are actually inversely correlated, consistent with prior findings that higher nitrite blocks SNO-Hb formation, as shown in FIG. 20. It follows that nitrite levels are unrelated to blood flow or tissue oxygenation.

Patients with PAD have well characterized microvascular dysfunction. When tested for recovery from transient ischemia of the lower limb, reperfusion in this cohort was significantly delayed, as shown in FIGS. 19A-E, and in all cases the t_{1/2} to reoxygenation was longer compared to the healthy controls. Importantly, while statistical differences in t_{1/2} to reoxygenation were not observed in other cohorts, a significant correlation was found between SNO-Hb levels and oxygenation rate across all patient cohorts. Taken together, with the genetic validation in mice, these results suggest that SNO-Hb is a key driver of blood flow autoregulation whereby tissue blood flow controls tissue oxygenation.

These findings have multiple clinical implications. Reactive hyperemia, previously viewed as a measure of endothelial function, has a significant RBC SNO component. More generally, endothelial NO and RBC SNO have different roles, the former in vascular health and the latter in tissue health. The results add to the body of research pointing to RBC SNO-Hb as a biomarker of tissue oxygenation status especially since SNO-Hb was directly correlated with reperfusion rate. Furthermore, the study shows that reactive hyperemia testing is a useful measure of SNO-Hb functionality in patient populations. The ability to enhance RBC SNO may improve tissue oxygenation and could have widespread clinical utility.

### Example: Measurement Device

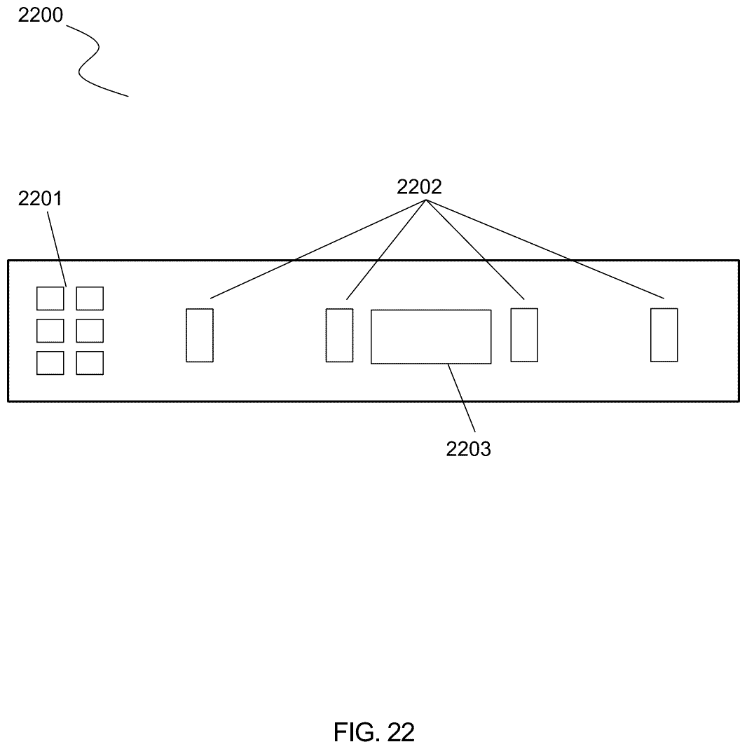

FIG. 22 illustrates an example measurement device 2200. The measurement device 2200, as described herein, can be wearable on a patient. The measurement device 2200 can include one or more light sources 2201 (e.g., light emitting diodes, organic light emitting diodes, lasers, or a singular light source producing multiple wavelengths). The one or more light sources 2201 can form an array. A control circuit and/or processor can control the one or more light sources 2201. For example, one or more of system 100 and/or system 200 may be embedded within, coupled to, or in communication with measurement device 2200. Accordingly, processor 112/214 can control the one or more light sources 2201. The one or more light sources 2201 may produce light at multiple wavelengths. As an example, the one or more light sources 2201 produce light at 525 nm, 650 nm, 750 nm, 810 nm, 850 nm, and 970 nm. In another example, at least one light source 2201 will produce green light, at least one light source will produce red light, and at least one light source will produce infrared light.

The measurement device 2200 can include one or more optical receivers 2202 (e.g., photodiodes or optical sensors). The one or more optical receivers 2202 can form an array. The one or more optical receivers 2202 can be placed a pre-determined distances from the one or more light sources 2201. The one or more optical receivers 2202 can function as one or more of sensor(s) 102/202 of system 100 and/or 200. Information received from the one or more optical receivers 2202 can be converted in an analog to digital converter. The measurement device 2200 can include one or more processors for processing the data from the one or more optical receivers 2202 (e.g., processor 112/214). The measurement device 2200 can include a communication interface (e.g., Bluetooth®, WiFi, 5G, etc.) for communicating data to external systems and/or processors. The measurement device 2200 can include one or more memory devices for storing data (e.g., computer readable medium 110/212).

The measurement device 2200 an include a display and/or audio output 2203 for presenting data on the device. Alternatively, data can be presented on an external system. For example, the measurement device 2200 can communicate, through the communication interface, to a personal computer or mobile device, and output to a user through the personal computer or mobile device. In this example, the personal computer or mobile device may function as system 100 and/or 200.

The measurement device 2200 can include one or more additional sensing elements including, but not limited to: a thermometer and a bio-impedance sensor, which may be included among sensor(s) 102/202. The data collected by the additional sensing elements can provide enhanced measurement and post-processing analysis.

Operation of the measurement device 2200 can include initiating a data capture sequence. A data capture sequence can include activating the one or more light sources 2201 in a timed sequence of turning on and off. A data capture sequence can include activating the one or more optical receivers 2202, at pre-determined distances, in conjunction with the activation of the one or more light sources 2201. Different wavelengths of generated light can be configured to target different substances within the body. Different pre-determined distances between an optical receiver 2202 and light source 2201 can target different depths within the body. As an example, green light (˜530 nm) can target pulse rate at a 3 mm distance. Red light (˜660 nm) can target deoxygenated hemoglobin, whereas infrared (˜940-980 nm) can target oxygenated hemoglobin at various depths of the body given the optical receiver's 2202 distance from the light source 2201. Infrared at 790-810 nm targets the isosbestic hemoglobin. The measurement device 2200 may capture the signals from the one or more optical receivers 2202. The captured signals can be preprocessed (i.e., run through an analog to digital converter and/or filtered). The captured signals can be converted into biomarkers. The biomarkers can be further processed, stored, and/or communicated (e.g., on device and/or to an external system).

### Example: Computer System

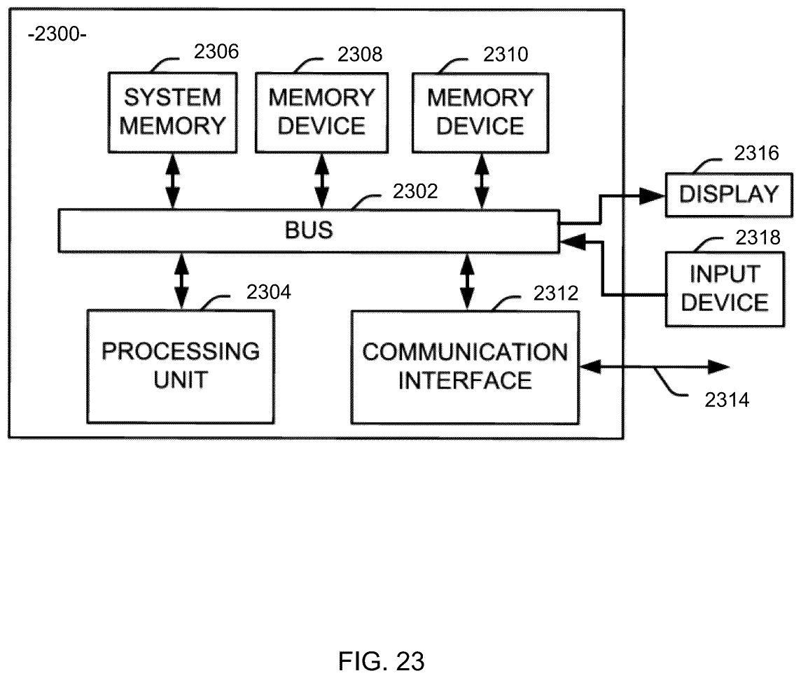

FIG. 23 is a schematic block diagram illustrating an example system 2300 of hardware components capable of implementing examples of the systems and methods disclosed herein. The system 2300 can include various systems and subsystems. The system 2300 can include one or more of a personal computer, a laptop computer, a mobile computing device, a workstation, a computer system, an appliance, an application-specific integrated circuit (ASIC), a server, a server BladeCenter, a server farm, etc.

The system 2300 can include a system bus 2302, a processing unit 2304, a system memory 2306, memory devices 2308 and 2310, a communication interface 2312 (e.g., a network interface), a communication link 2214, a display 2316 (e.g., a video screen), and an input device 2318 (e.g., a keyboard, touch screen, and/or a mouse). The system bus 2302 can be in communication with the processing unit 2304 and the system memory 2306. The additional memory devices 2308 and 2310, such as a hard disk drive, server, standalone database, or other non-volatile memory, can also be in communication with the system bus 2302. The system bus 2302 interconnects the processing unit 2304, the memory devices 2306 and 2310, the communication interface 2312, the display 2316, and the input device 2318. In some examples, the system bus 2302 also interconnects an additional port (not shown), such as a universal serial bus (USB) port.

The processing unit 2304 can be a computing device and can include an application-specific integrated circuit (ASIC). The processing unit 2304 executes a set of instructions to implement the operations of examples disclosed herein. The processing unit can include a processing core.

The additional memory devices 2306, 2308, and 2310 can store data, programs, instructions, database queries in text or compiled form, and any other information that may be needed to operate a computer. The memories 2306, 2308 and 2310 can be implemented as computer-readable media (integrated or removable), such as a memory card, disk drive, compact disk (CD), or server accessible over a network. In certain examples, the memories 2306, 2308 and 2310 can comprise text, images, video, and/or audio, portions of which can be available in formats comprehensible to human beings.

Additionally, or alternatively, the system 2300 can access an external data source or query source through the communication interface 2312, which can communicate with the system bus 2302 and the communication link 2314.

In operation, the system 2300 can be used to implement one or more parts of a system in accordance with the present invention, such as system 100, system 200, and/or measurement device 2200. Computer executable logic for implementing the diagnostic system resides on one or more of the system memory 2306, and the memory devices 2308 and 2310 in accordance with certain examples. The processing unit 2304 executes one or more computer executable instructions originating from the system memory 2306 and the memory devices 2308 and 2310. The term “computer readable medium” as used herein refers to a medium that participates in providing instructions to the processing unit 2304 for execution. This medium may be distributed across multiple discrete assemblies all operatively connected to a common processor or set of related processors.

Specific details are given in the above description to provide a thorough understanding of the embodiments. However, it is understood that the embodiments can be practiced without these specific details. For example, physical components can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes but is not limited to portable or fixed storage devices, optical storage devices, wireless channels, and/or various other storage mediums capable of storing that contain or carry instruction(s) and/or data.

The invention includes the following embodiments:
1. A system comprising:
at least one sensor configured to non-invasively measure a biometric parameter within a region of interest of a subject and provide a time series of measurements of the biometric parameter;
at least one processor in communication with the sensor; and
at least one non-transitory computer readable medium storing machine-readable instructions that, when executed by the at least one processor, cause the at least one processor to perform processing comprising:
receiving the time series of measurements of the biometric parameter;
generating, using a predictive model, a value representing an endogenous S-nitrosothiol content of tissue within the region of interest from the time series of measurements of the biometric parameter; and
providing, by a user interface, the value representing the endogenous S-nitrosothiol content of tissue within the region of interest to a user.
2. The system of claim 1, wherein the at least one sensor includes a near-infrared spectroscopy sensor.
3. The system of claim 2, wherein the at least one sensor includes a Fourier transform infrared spectrometer.
4. The system of claim 1, wherein the at least one sensor includes a sensor configured to non-invasively measure oxygen saturation and blood volume within the region of interest and provides a time series of oxygen saturation measurements and a time series of blood volume measurements.
5. The system of claim 4, wherein the generating, using the predictive model, comprises:
determining a linearity of relationship between the time series of blood volume measurements and the time series of oxygen saturation measurements and provides a set of parameters; and
generating the value representing the endogenous S-nitrosothiol content of tissue within the region of interest from the set of parameters.
6. The system of claim 5, wherein the generating, using the predictive model, comprises:
using a linear regression model to provide a best-fit line defined by the set of parameters, the set of parameters including a slope of the best-fit line; and
generating the value representing the endogenous S-nitrosothiol content of tissue within the region of interest from the slope of the best-fit line.
7. The system of claim 5, wherein:
the processing further comprises measuring an overshoot response in one of blood flow and oxygen saturation above baseline following one of a physiological occlusion of blood flow to the region of interest and/or an external occlusion of blood flow to the region of interest, thereby providing one of the time series of oxygen saturation measurements and the time-series of blood volume measurements; and
the generating, using the predictive model, comprises using the rate or overshoot value, thereby generating a value representing the endogenous S-nitrosothiol content of tissue within the region of interest.
8. The system of claim 1, wherein the sensor is configured to non-invasively measure the biometric parameter within the region of interest during one of a period of exercise by the subject and a time period immediately after the period of exercise by the subject.
9. A method comprising:
non-invasively measuring, by at least one sensor, a biometric parameter within a region of interest of a subject;
providing, by the at least one sensor, a time series of measurements of the biometric parameter;
generating, by at least one processor in communication with the at least one sensor, a value representing an endogenous S-nitrosothiol content of tissue within the region of interest from the time series of measurements of the biometric parameter; and
storing, by the at least one processor, the value representing the endogenous S-nitrosothiol content of tissue within the region of interest in a non-transitory computer readable medium.
10. The method of claim 9, wherein the measuring comprises non-invasively measuring blood volume and oxygen saturation within the region of interest to provide a time series of oxygen saturation measurements and a time series of blood volume measurements.
11. The method of claim 10, wherein non-invasively measuring blood volume and oxygen saturation within the region of interest of the subject comprises measuring blood volume and oxygen saturation within the region of interest during exercise by the subject.
12. The method of claim 10, wherein non-invasively measuring blood volume and oxygen saturation within the region of interest of the subject comprises measuring blood volume and oxygen saturation within the region of interest immediately after one of a physiological occlusion of blood flow to the region of interest and an external occlusion of blood flow to the region of interest.
13. The method of claim 10, wherein:
the region of interest comprises muscle tissue;
non-invasively measuring blood volume comprises measuring a total hemoglobin metric within the muscle tissue to provide the time series of blood volume measurements as a time series of total hemoglobin metrics; and
the generating comprises using a predictive model to generate the value representing endogenous S-nitrosothiol content of the muscle tissue from the time series of total hemoglobin measurements and the time series of oxygen saturation measurements.
14. The method of claim 10, wherein generating the value representing the endogenous S-nitrosothiol content of tissue within the region of interest from the time series of oxygen saturation measurements and the time series of blood volume measurements comprises determining a linearity of relationship between the time series of blood volume measurements and the time series of oxygen saturation measurements.
15. The method of claim 14, wherein determining the linearity of relationship between the time series of blood volume measurements and the time series of oxygen saturation measurements comprises fitting the time series of blood volume measurements and the time series of oxygen saturation measurements to a linear regression model to provide a best-fit line, the value representing the endogenous S-nitrosothiol content of tissue within the region of interest being derived from one of a slope of the best-fit line and a correlation coefficient between oxygen saturation and blood volume.
16. The method of claim 10, wherein:
the value representing the endogenous S-nitrosothiol content of tissue within the region of interest includes a first value representing the endogenous S-nitrosothiol content of tissue within the region of interest;
the time series of blood volume measurements includes a first time series of blood volume measurements; and
the time series of oxygen saturation measurements includes a first time series of oxygen saturation measurements, the method further comprising:
non-invasively measuring, by the at least one sensor, blood volume and oxygen saturation within the region of interest of the subject;
providing, by the at least one sensor, a second time series of oxygen saturation measurements and a second time series of blood volume measurements;
generating, by the at least one processor, a second value representing the endogenous S-nitrosothiol content of tissue within the region of interest from the second time series of oxygen saturation measurements and the second time series of blood volume measurements using a predictive model; and
comparing, by the at least one processor, the first value representing the endogenous S-nitrosothiol content of tissue within the region of interest and second value representing the endogenous S-nitrosothiol content of tissue within the region of interest, wherein a result of the comparing indicates an effectiveness of a therapy provided to the patient.
17. The method of claim 10, further comprising generating, by the at least one processor, a personalized nitric oxide metric from the value representing the endogenous S-nitrosothiol content of tissue within the region of interest.
18. The method of claim 10, further comprising generating, by the at least one processor, a usable oxygen consumption (UO2) metric from the value representing the endogenous S-nitrosothiol content of tissue within the region of interest.
19. The method of claim 18, further comprising deducing, by the at least one processor, the origin of a change in UO2, to represent a limitation in one of oxygen supply and oxygen utilization.
20. The method of claim 18, wherein generating the UO2 metric from the value representing the endogenous S-nitrosothiol content of tissue within the region of interest comprises generating the UO2 metric in real time.
21. The method of claim 10, further comprising generating, by the at least one processor, a maximum nitric oxide power metric from the value representing the endogenous S-nitrosothiol content of tissue within the region of interest.
22. The method of claim 21, wherein generating the maximum nitric oxide power metric from the value representing the endogenous S-nitrosothiol content of tissue within the region of interest comprises generating the maximum nitric oxide power metric in real time.
23. The method of claim 10, further comprising generating, by the at least one processor, a maximum nitric oxide endurance metric from the value representing the endogenous S-nitrosothiol content of tissue within the region of interest.
24. The method of claim 23, wherein generating the maximum nitric oxide endurance metric from the value representing the endogenous S-nitrosothiol content of tissue within the region of interest comprises generating the maximum nitric oxide endurance metric in real time.
25. The method of claim 10, wherein generating the value representing the endogenous S-nitrosothiol content of tissue within the region of interest comprises the value representing the endogenous S-nitrosothiol content of tissue within the region of interest in real time.
26. A method comprising:
non-invasively measuring, by at least one sensor, blood volume and oxygen saturation within a region of interest of a subject;
providing, by the at least one sensor, a time series of oxygen saturation measurements and a time series of blood volume measurements;
determining, by at least one processor in communication with the at least one sensor, a linear relationship between the time series of blood volume measurements and the time series of oxygen saturation measurements via a predictive model;
generating, by the at least one processor, a value representing an endogenous S-nitrosothiol content of tissue within the region of interest from the time series of oxygen saturation measurements and the time series of blood volume measurements from the determined linear relationship; and
storing, by the at least one processor, the value representing the endogenous S-nitrosothiol content of tissue within the region of interest in a non-transitory computer readable medium.
27. The method of claim 26, wherein non-invasively measuring blood volume and oxygen saturation within the region of interest of the subject comprises measuring blood volume and oxygen saturation within the region of interest one of during a period of exercise by the subject or after the period of exercise.
28. The method of claim 26, wherein determining the linearity of relationship between the time series of blood volume measurements and the time series of oxygen saturation measurements comprises fitting the time series of blood volume measurements and the time series of oxygen saturation measurements to a linear regression model to provide a best-fit line, the value representing the endogenous S-nitrosothiol content of tissue within the region of interest being derived from a slope of the best-fit line.
29. The method of claim 26, wherein non-invasively measuring blood volume and oxygen saturation within the region of interest of the subject comprises measuring blood volume and oxygen saturation within the region of interest via near-infrared spectroscopy.
30. The method of claim 26, further comprising generating, by the at least one processor, at least one of a maximum nitric oxide endurance metric, a maximum nitric oxide power metric, a usable oxygen consumption metric, and a personalized nitric oxide metric from the value representing the endogenous S-nitrosothiol content of tissue within the region of interest.
31. The method of claim 26, wherein:
non-invasively measuring blood volume and oxygen saturation comprises determining a rate or overshoot in one of oxygen saturation and blood volume following one of exercise or occlusion; and
determining the linear relationship between the time series of blood volume measurements and the time series of oxygen saturation measurements comprises providing a best-fit value representing the endogenous S-nitrosothiol content of tissue within the region of interest;
the method further comprising measuring, by the at least one processor, at least one of a personalized nitric oxide measurement, a UO2 value, a max nitric oxide endurance, and a max nitric oxide power from the best-fit value.
32. The method of claim 26, further comprising:
providing, by the at least one processor, the value representing the endogenous S-nitrosothiol content of tissue within the region of interest to a user; and
assigning, by the at least one processor, exercises to the subject to increase the S-nitrosothiol levels in blood.
33. The method of claim 26, further comprising deriving, by the at least one processor a measure of at least one of health, performance, and fitness, from the value representing the endogenous S-nitrosothiol content of tissue within the region of interest.
34. The method of claim 26, further comprising:
deriving, by the at least one processor, a value representing a value representing a UO2 content of tissue from the value representing the endogenous S-nitrosothiol content of tissue within the region of interest; and
deriving, by the at least one processor, a measure of one of at least one of health, performance, and fitness, from the value representing the UO2 content of tissue within the region of interest.
35. The method of claim 26, wherein the value representing the endogenous S-nitrosothiol content of tissue within the region of interest represents the S-nitrosothiol released by hemoglobin within the region of interest.
36. The method of claim 35, wherein the value of endogenous S-nitrosothiol is predictive of the rate of reoxygenation of the tissue.
37. The system of claim 7, wherein the endogenous S-nitrosothiol content of tissue within the region of interest is predictive of the rate of reoxygenation of the tissue.
38. The method of claim 18, further comprising determining, by the at least one processor, that the UO2 measurement is low relative to a subject not having dementia, thereby indicating a risk of dementia or loss of cognitive function.
39. The method of claim 38, wherein the dementia or loss of cognitive function is associated with Alzheimer's Disease.
40. The method of claim 16, further comprising:
generating, by the at least one processor, a first UO2 measurement from the value representing the endogenous S-nitrosothiol content of tissue within the region of interest;
generating, by the at least one processor, a second UO2 measurement from the second value representing the endogenous S-nitrosothiol content of tissue within the region of interest;
determining, by the at least one processor, that the first UO2 measurement is low relative to a subject not having dementia, thereby indicating a dementia or loss of cognitive function; and
determining, by the at least one processor, that an increase in the second UO2 measurement relative to the first UO2 measurement is indicative of an improvement in the dementia or cognitive function in the subject.
41. The method of claim 40, wherein the dementia or loss of cognitive function is associated with Alzheimer's Disease.

In the preceding description, specific details have been set forth in order to provide a thorough understanding of example implementations of the invention described in the disclosure. However, it will be apparent that various implementations may be practiced without these specific details. For example, circuits, systems, networks, processes, and other components may be shown as components in block diagram form in order not to obscure the example implementations in unnecessary detail. In other instances, well-known circuits, processes, algorithms, structures, and techniques may be shown without unnecessary detail in order to avoid obscuring the examples. The description of the example implementations will provide those skilled in the art with an enabling description for implementing an example of the invention, but it should be understood that various changes may be made in the function and arrangement of elements without departing from the spirit and scope of the invention. Accordingly, the present invention is intended to embrace all such alterations, modifications, and variations that fall within the scope of the appended claims.

Although the invention has been described with reference to the above examples, it will be understood that modifications and variations are encompassed within the spirit and scope of the invention. Accordingly, the invention is limited only by the following claims.

What is claimed is:

1. A system comprising:
at least one sensor configured to non-invasively capture a plurality of oxygen saturation measurements within a region of interest of a subject over a period of time and to non-invasively capture a plurality of blood volume measurements within the region of interest over the period of time;
at least one processor in communication with the sensor; and
at least one non-transitory computer readable medium storing machine-readable instructions that, when executed by the at least one processor, cause the at least one processor to perform processing comprising:
receiving the plurality of oxygen saturation measurements and the plurality of blood volume measurements;
generating, using a predictive model, a value representing an endogenous S-nitrosothiol content of tissue within the region of interest from the plurality of oxygen saturation measurements and the plurality of blood volume measurements; and
providing, by a user interface, the value representing the endogenous S-nitrosothiol content of tissue within the region of interest to a user.

2. The system of claim 1, wherein the at least one sensor includes a near-infrared spectroscopy sensor.

3. The system of claim 2, wherein the at least one sensor includes a Fourier transform infrared spectrometer.

4. The system of claim 1, wherein the generating, using the predictive model, comprises:
determining a linearity of relationship between the plurality of blood volume measurements and the plurality of oxygen saturation measurements; and
generating the value representing the endogenous S-nitrosothiol content of tissue within the region of interest from the linearity.

5. The system of claim 4, wherein the generating, using the predictive model, comprises:
using a linear regression model to provide a best-fit line having a slope; and
generating the value representing the endogenous S-nitrosothiol content of tissue within the region of interest from the slope of the best-fit line.

6. The system of claim 4, wherein:
the processing further comprises measuring an overshoot response in one of blood flow and oxygen saturation above baseline following one of a physiological occlusion of blood flow to the region of interest and/or an external occlusion of blood flow to the region of interest, thereby providing one of the plurality of oxygen saturation measurements and the plurality of blood volume measurements; and
the generating, using the predictive model, comprises using the rate or overshoot value, thereby generating a value representing the endogenous S-nitrosothiol content of tissue within the region of interest.

7. A method comprising:
non-invasively capturing, by at least one sensor, a plurality of oxygen saturation measurements within a region of interest of a subject over a period of time;
non-invasively capturing, by the at least one sensor, a plurality of blood volume measurements within the region of interest over the period of time;
generating, by at least one processor in communication with the at least one sensor, a value representing an endogenous S-nitrosothiol content of tissue within the region of interest from the plurality of oxygen saturation measurements and the plurality of blood volume measurements; and
storing, by the at least one processor, the value representing the endogenous S-nitrosothiol content of tissue within the region of interest in a non-transitory computer readable medium.

8. The method of claim 7, wherein non-invasively capturing the plurality of blood volume measurements and non-invasively capturing the plurality of oxygen saturation measurements comprises measuring blood volume and oxygen saturation within the region of interest during exercise by the subject.

9. The method of claim 7, wherein non-invasively capturing the plurality of blood volume measurements and non-invasively capturing the plurality of oxygen saturation measurements comprises measuring blood volume and oxygen saturation within the region of interest immediately after one of a physiological occlusion of blood flow to the region of interest and an external occlusion of blood flow to the region of interest.

10. The method of claim 7, wherein:
the region of interest comprises muscle tissue;
non-invasively capturing the plurality of blood volume measurements comprises measuring a total hemoglobin metric within the muscle tissue; and
the generating comprises using a predictive model to generate the value representing endogenous S-nitrosothiol content of the muscle tissue from the measured total hemoglobin metric and the plurality of oxygen saturation measurements.

11. The method of claim 7, wherein generating the value representing the endogenous S-nitrosothiol content of tissue within the region of interest from the plurality of blood volume measurements and the plurality of oxygen saturation measurements comprises determining a linearity of relationship between the plurality of blood volume measurements and the plurality of oxygen saturation measurements, wherein fitting the plurality of blood volume measurements and the plurality of oxygen saturation measurements to a linear regression model provides a best-fit line, the value representing the endogenous S-nitrosothiol content of tissue within the region of interest being derived from one of a slope of the best-fit line and a correlation coefficient between oxygen saturation and blood volume.

12. The method of claim 7, wherein the value representing the endogenous S-nitrosothiol content of tissue within the region of interest includes a first value representing the endogenous S-nitrosothiol content of tissue within the region of interest, the method further comprising:
non-invasively capturing, by the at least one sensor, a second plurality of oxygen saturation measurements within a region of interest of a subject over a second period of time;
non-invasively capturing, by the at least one sensor, a second plurality of blood volume measurements within the region of interest over the second period of time;
generating, by the at least one processor, a second value representing the endogenous S-nitrosothiol content of tissue within the region of interest from the second plurality of oxygen saturation measurements and the second plurality of blood volume measurements using a predictive model; and
comparing, by the at least one processor, the first value representing the endogenous S-nitrosothiol content of tissue within the region of interest and second value representing the endogenous S-nitrosothiol content of tissue within the region of interest, wherein a result of the comparing indicates an effectiveness of a therapy provided to the patient.

13. The method of claim 7, further comprising generating, by the at least one processor, a personalized nitric oxide metric from the value representing the endogenous S-nitrosothiol content of tissue within the region of interest.

14. The method of claim 7, further comprising:
generating, by the at least one processor, a usable oxygen consumption (UO2) metric from the value representing the endogenous S-nitrosothiol content of tissue within the region of interest; and
deducing, by the at least one processor, the origin of a change in UO2, to represent a limitation in one of oxygen supply and oxygen utilization.

15. The method of claim 7, further comprising generating, by the at least one processor, a maximum nitric oxide power metric from the value representing the endogenous S-nitrosothiol content of tissue within the region of interest.

16. The method of claim 7, further comprising generating, by the at least one processor, a maximum nitric oxide endurance metric from the value representing the endogenous S-nitrosothiol content of tissue within the region of interest.
